(19) United States
(12) Patent Application Publication
(10) Pub. No.: US 2025/0312569 A1
(43) Pub. Date: Oct. 9, 2025

(54) RAPID EXCHANGE CATHETER

(71) Applicant: Radial1, Inc., Teaneck, NJ (US)

(72) Inventor: Daniel Ezra Walzman, Teaneck, NJ (US)

(73) Assignee: Radial1, Inc., Teaneck, NJ (US)

(21) Appl. No.: 18/668,492

(22) PCT Filed: Dec. 2, 2022
(86) PCT No.: PCT/US2022/051599

§ 371 Date: May 20, 2024

Related U.S. Application Data
(60) Provisional application No. 63/286,169, filed on Dec. 6, 2021.

(51) Int. Cl. A61M 25/01; A61M 25/00; A61M 25/06; A61M 25/09; A61M 39/00; A61M 39/02

(52) U.S. Cl. CPC A61M 25/0172; A61M 25/0045; A61M 25/0052; A61M 25/0102; A61M 25/0136; A61M 25/0147; A61M 39/0247; A61M 2025/0183; A61M 25/065; A61M 2025/09116; A61M 2039/0036; A61M 2039/0258; A61M 2039/027

(57) ABSTRACT

A catheter having a proximal opening, a distal opening, a lumen extending through the catheter and a side opening forming a rapid exchange port dimensioned and configured for insertion of a guiding device through the side opening. The catheter includes a seal movable between a closed position to reduce leakage through the side opening and an open position, wherein the seal is movable to an open position by the guiding device. The catheter can have one or more independently deflectable zones.

### BACKGROUND

This application claims priority to provisional application 63/286,169 filed on Dec. 6, 2021, the entire contents of which are incorporated herein by reference.

### TECHNICAL FIELD

This application relates generally to endovascular devices and more particularly to rapid exchange catheters and their method of use.

### BACKGROUND OF RELATED ART

The advantages of minimally invasive treatments are well known and include reduced trauma, shorter hospital time, faster patient recovery, reduced cost, etc. In minimally invasive endovascular procedures, access to the treatment site is typically obtained through a remote access, e.g., via the femoral artery, brachial artery, radial artery, etc. and the catheter is guided over a guidewire to the target site. Typically, the guidewire is inserted through the opening of the catheter and the catheter is threaded over its entire length over the guidewire. An alternative method utilizes rapid exchange catheters. In such rapid exchange catheters, the distal opening for the guidewire is closer to the distal end of the catheter so only a portion of the catheter is threaded over the guidewire. This is increasingly popular for intravascular catheter treatments such as coronary and carotid artery stenting.

Access to patient blood vessels is necessary for a wide variety of medical, diagnostic, and/or therapeutic purposes. While a wide variety of variations exist, the basic technique often relies on access via a long and tortuous path. Craniofacial angiography and cardiac catheterization, for example, are often performed through a transfemoral route. More recently, however, a trans-radial approach has been developed for cardiac catheterization and become more common. Trans-radial for medical purposes means through, by way of, or employing the radial artery as an access site, typically percutaneously. More specifically, trans-radial access is used to perform medical catheterization procedures, therapeutic procedures as well as other procedures. Recently, trans-radial access is being used for more craniofacial and peripheral procedures as well. A need exists for new catheters and devices tailored for such use, to optimize safety and efficacy.

Trans-radial access for medical intervention has shown significant reduction in trauma and blood loss, even with aggressive use of anticoagulation and antiplatelet therapies, compared to trans-femoral procedures. Often during such procedures, patients are given high doses of blood thinners and platelet inhibiting medications. Some studies have demonstrated that trans-radial vascular access has lower access-site major complication rates than transfemoral access. Recovery times for the patient are also shorter. However, current catheter technology can complicate trans-radial access to contralateral carotid, vertebral circulations, and ipsilateral carotid circulations in certain patients. There is also difficulty in selectively accessing the contralateral internal mammary artery, which is often important in cardiac procedures in patients with prior bypass surgery, as well as in selectively accessing many other vessels via a trans-radial route.

With the introduction of a greater number and variety of intravascular techniques, including angioplasty, atherectomy, endovascular aneurysm repair, thrombectomy, minimally invasive cardiac surgery, and the like, a need has arisen to improve access to the target vascular site.

The catheters need to enable access to all “great vessels” of the aortic arch while providing an inner lumen through which additional wires and catheters can be advanced into all “selective cerebral angiography vessels (bilateral internal carotid artery, bilateral vertebral artery, bilateral external carotid artery) and even beyond—into the brain and head and neck for interventions.

Radial artery access is known in the art and is typically achieved with a short, bevel 21-gauge needle, and typically, a 0.018-0.021 guide wire. This smaller needle system allows for better control and pulsatile blood flow can be seen immediately. It is suggested during a radial artery catheterization to use a smaller needle than one traditionally used during femoral catheterization, which may reduce difficulty when obtaining access.

There are catheters on the market by various vendors designed specifically for radial artery access. These catheters for cardiac use have the common characteristic of a primary and secondary curve. A radial-specific catheter enables angiography of both right and left coronaries with a clockwise and counterclockwise rotation of one catheter. Eliminating catheter exchange can result in less total procedure time as well as fluoroscopy time and less incidence of radial artery spasm.

In using the radial access approach, oftentimes the radial artery has severe spasms. In order to reduce such spasms, it is desirable to inject a “radial cocktail” which typically consists of a mixture of verapamil, nitroglycerin, and heparin which may optionally be diluted with fluid for injection. Such cocktail in addition to reducing spasms can reduce and/or prevent thrombosis.

However, if catheters of full length are used without an outer sheath and placed over a wire beyond the radial artery, the radial cocktail cannot be injected since the opening in the catheter is already past the radial artery. If a 100 cm sheath introducer is used, it also will be placed past the radial artery if introduced through its full length. Introducing it a shorter length would still require using a wire greater than the full length of the catheter, which would add complexity and would be less user friendly than a short wire, which can be more easily handled by a single operator.

Currently, a short guidewire (40-50 cm) is typically first introduced, and a short sheath, often 10-11 cm, is placed in the radial artery. The radial cocktail is then administered. However, if larger diameter long sheaths are desired that are of a similar diameter to the larger short radial sheaths, then placement of a short radial sheath is an extra step that can add time and cost, and may increase the risk of injury to the patient. Alternatively, a long guidewire, e.g., about 140 cm-150 cm can be utilized to gain access to the vasculature. The guidewire is inserted via a needle, and then the needle is removed, and the catheter with dilator is inserted over the guidewire to the target site. The dilator and wire are then removed, leaving the catheter in place. There are disadvantages of utilizing the long guidewire as the long guidewire is not user friendly, is cumbersome and sometimes requires two clinicians for insertion. Additionally, if the catheter distal end is not first placed in the radial artery, the opportunity to deliver the desired “radial cocktail” may be lost, and unwanted vascular spasm may occur.

It would be advantageous to provide a system and method that avoids the disadvantages of the long guidewire.

Fluid could leak out of the side hole (port) of the rapid exchange catheter, if the rapid exchange lumen is shared with the main lumen of the sheath/catheter. It would be advantageous to prevent leaking of fluid through the side port. If the rapid exchange lumen is independent of the main lumen of the sheath/catheter, then a larger outer diameter catheter is needed for a given inner diameter of the main lumen of the sheath/catheter, which limits the utility of the sheath/catheter for many uses, especially within the limited confines of safe access via a radial artery, which is typically significantly smaller in diameter than the femoral artery. It would be advantageous to provide a system and method that addresses such fluid leakage.

Radial access catheters by the inventor of the present application which can make safe percutaneous access of either carotid artery feasible in the vast majority of patients, are disclosed for example in U.S. application Ser. Nos. 16/013,707, filed Jun. 20, 2018 (U.S. Pat. No. 10,258,371), 16/575,302, filed Sep. 18, 2019 (Publication No. 2020/0009351), 16/600,096, filed Oct. 11, 2019 (Publication No. 2020/0060723), 16/602,469, filed Oct. 11, 2019 (Publication No. 2020-0078554), and 17/423,502, filed Jul. 16, 2021 (Publication No. 2022-0118219). The entire contents of each of these applications are incorporated by reference herein. The catheters disclosed therein may also reduce access-site complications.

Most often when a sheath/catheter is introduced into a vessel percutaneously, often over a wire, the catheter is provided in combination with a dilator positioned within the lumen of the catheter, which is longer than the catheter and extends beyond the catheter on the distal end, and often on the proximal end as well. The dilator is preferably stiffer than the catheter, and helps introduction of the catheter through the skin into a vessel while limiting potential damage to the vessel.

Currently, an introducer sheath is typically utilized to gain initial access to the vessel. The introducer sheath however needs to have a larger diameter than the catheter being inserted. Additionally, it requires an additional more expensive component. Additionally, current catheters designed for trans-radial use typically have a fixed shape, which can limit access across tortuous anatomy, especially when navigating from one arm to vessels in the opposite side of the body. It would be advantageous to address these deficiencies of the introducer sheath.

It would also be advantageous to provide such radial access catheters with deflectable/steerable distal sections to facilitate arterial access.

### SUMMARY OF THE INVENTION

The present invention overcomes the problems and deficiencies of the prior art.

In one aspect of the present invention, a catheter is provided comprising a proximal opening, a distal opening, a lumen extending through the catheter and a side opening (side hole) forming a rapid exchange port dimensioned and configured for insertion of a guiding device through the side opening. The catheter's side opening optionally includes a seal movable between a closed position to reduce leakage through the side opening and an open position, wherein the seal is movable to an open position by the guiding device. The side hole is preferably within 20 cm of the distal end hole, but alternate distances are also contemplated. In some embodiments, the side opening is spaced between about 3 centimeters and about 15 centimeters from the distal opening of the catheter, although other dimensions are also contemplated.

In some embodiments, the guiding device is a guidewire.

In some embodiments, the seal comprises a tube having a side opening and connecting a first portion of the catheter with a second portion of the catheter and an elastic material is placed over the tube, wherein the guiding device presses against the elastic material to move the elastic material to provide a gap to exit the side opening of the catheter.

In some embodiments, the seal comprises a flap movably positioned over the side opening of the catheter and movable by the guiding device for exit of the guiding device through the side opening. In some embodiments, a band is positioned on the catheter and an attachment element attaches the flap to the band, wherein the guiding device presses the flap outwardly away from the side opening to create a gap for exit from the lumen through the side opening.

In some embodiments, tube has a band portion and a flap is connected to the band portion, the tube positioned in the side opening.

In some embodiments, the catheter includes an inner polymeric liner movable by the guiding device to create a gap for exit of the guiding device through the side opening.

In some embodiments the seal is substantially impermeable to blood and other fluids. In other embodiments the seal is semipermeable. In other embodiments the seal is only semipermeable above a certain pressure. In a preferred embodiment, there is less than 10% leakage during injection at typical pressures.

In some embodiments, the catheter comprises a proximal body portion and a distal body portion, and the proximal body portion is connected to the distal body portion by a connecting tube, and the seal is formed in the connecting tube. In some embodiments, the proximal body portion comprises a braided tube and the distal body portion includes a coil. In some embodiments, an elastic material is positioned over the opening in the connecting tube, a portion of the elastic material movable by the guiding device to create a gap for exit of the guiding device from the lumen.

In some embodiments, the seal is formed in a tube positioned in the lumen of the catheter, the tube having a coil embedded in the wall.

In some embodiments, the guiding device extends through a dilator positioned in the lumen of the catheter. The dilator extends within the catheter lumen and can extend proximal to the proximal end of the catheter, distal to the distal end of the catheter or extend both distal to the distal end of the catheter and proximal to the proximal end of the catheter.

In some embodiments, the catheter has a distal deflection zone. The zone can be deflectable by a pull ring and a pull wire. Alternatively, it can be deflected by a push wire. An independent proximal deflection zone can also be provided, deflectable by a pull ring and pull wire, or alternatively by a push wire. The rings and wires can be substantially within a wall of the catheter. The wires can be offset from a longitudinal axis of the catheter.

The present invention also provides in accordance with another aspect, the catheter as disclosed herein in combination with a dilator as disclosed herein positioned within the lumen of the catheter and having a distal end hole and a lumen in at least its distal segment extending distally from the distal end hole. The inner lumen extends at least until a side hole in the dilator. A guiding surface is adjacent the side opening of the catheter, the guiding surface guiding the guiding device toward the side opening. In some embodiments, the guiding surface comprises a ramped surface angled away from the longitudinal axis toward the side opening. The dilator is preferably stiffer than the catheter, and helps the catheter be introduced through the skin into a vessel while limiting potential damage to the vessel.

In the embodiments wherein the dilator includes a ramp, the proximal end of the guiding device, e.g., wire, is directed out of the side hole via the ramp, the ramp positioned adjacent the side hole in the catheter. In some embodiments, the cover provides a seal to reduce leakage therethrough, and the guiding device is forced against the seal to move the seal to extend outwardly through the side hole.

In some embodiments, the dilator lumen extends proximal to the ramp; in other embodiments there is no lumen or smaller lumen proximal to the ramp, with the dilator extending proximally beyond the proximal end of the catheter but having no inner lumen along its proximal segment.

In some alternate embodiments the catheters disclosed herein do not include a seal. The present invention also provides in some embodiments a steerable catheter with at least one end hole at its distal end which enters the patient, and at least one end hole at its proximal end which remains outside the patient and which the treating practitioner retains access to.

In a preferred embodiment, there is a single distal end hole and a single proximal end hole. The proximal end hole may have a hub attached, which optionally has a luer-lock to which other components can be affixed. Alternatively, the hub may have a diaphragm or other components.

The steerable catheter can have at least one active steering (steerable) zone within the distal 25 cm of the catheter, with at least one independent controller near the proximal end of the catheter that controls each steering zone independently. The controllers remain outside the patient. In a preferred embodiment, there are two independent steering (deflection) zones along the distal 25 cm of the catheter, and two corresponding controllers near the proximal end of the device, one for each steering zone. Each steering zone, in some embodiments, is controlled (regulated) by at least one wire situated substantially within the wall of the catheter. In some embodiments, one steering zone may be controlled by one wire, and another steering zone may be controlled by at least two wires. One or more shape memory alloy or polymer may also alternatively be used to control a steering zone or zones.

The present invention in some embodiments includes at least one circumferential marker ring, which can optionally be radiopaque. In some embodiments, the distal end of a steerable wire may be attached/inserted into the marker ring. Some marker rings may be completely circumferential. Some marker rings may have at least one gap. Any gap may allow passage of a wire past the marker ring while minimizing the wall thickness of the catheter at that site.

The controller can be a wheel or a lever; but other controller mechanisms are also envisioned. The controller may directly attach to a wire, or may control a gear attached to a wire, or may otherwise indirectly control a wire or shape memory polymer via a signal. Each steering zone is preferably between about 1 cm to about 10 cm long, although other lengths are also contemplated.

In some embodiments, the wire is shortened or pulled, the catheter deflects toward the wire at its distal insertion/attachment site, and when the wire is lengthened or pushed, the catheter deflects away from the wire at the wire's distal insertion/attachment site. Alternatively, when the wire is lengthened or pushed the catheter deflects toward the wire at the distal insertion/attachment site and when shortened or pushed deflects away from the wire. In some embodiments, the wire has a neutral position and is pushed from its neutral position to bend in one direction and pulled from its neutral position to bend in the opposite direction. Regions of varying rigidity and flexibility along the catheter further contribute to defining steering zones and their function. In embodiments where a single wire is used to steer a single steering zone, the controller effectively pulls or pushes that wire near its proximal end. In embodiments where two opposite wires on either side of the catheter control each steering zone, each controller may simultaneously pull on one wire while pushing on the other wire that governs that zone. The opposite motion of the controller would deflect the catheter in the opposite direction. Some embodiments can have multiple independent steering zones, and each zone may independently be controlled by a differing wire(s) arrangement.

Radio-opaque markers can optionally be provided at the distal end of each steering zone, at the proximal end of each steering zone and/or near the distal end of the catheter. Additional radiopaque markers may be provided as well.

The present invention includes methods to use the catheter and devices disclosed herein to access for example bilateral vertebral arteries, bilateral internal carotid arteries, and bilateral vertebral arteries selectively via a unilateral percutaneous arm access in most patients, optionally via a radial artery access site. Optionally a needle is used to percutaneously access a vessel, and a wire is passed through the needle into a vessel such as a right radial artery, and then the needle removed leaving the wire in place. Alternatively, an introducer catheter can be passed over the needle, as is more commonly used with a double wall puncture technique, and the needle then removed. The wire can then be advanced into the vessel through the introducer catheter and the introducer catheter can then be removed, leaving the wire in the vessel. In either scenario, a steerable catheter with a dilator within it can then be advanced over the wire into the radial artery.

In a preferred embodiment, the catheter has a side hole about 3 cm to about 15 cm from the distal end, optionally with a rescaling seal over it, and the dilator has a ramp and side hole aligned with the catheter side hole. During use, the wire and dilator are removed, and a “radial cocktail,” which may contain one or more vasodilator drugs and one or more blood thinner, is injected through the proximal hole of the catheter and at least mostly into the radial artery. The catheter can then be advanced, optionally over another catheter and wire, and under fluoroscopic guidance. When desired, the inner catheter and wire can be partly or fully withdrawn proximal to one or more steering zones of the catheter, and the steering zone can be steered to a desired shape to help direct it to a desired vessel.

In one method (technique), the catheter can be advanced from a right radial artery into the aortic arch and its distal end into the descending aorta. The catheter can then be steered and moved to engage the origin of the subclavian artery. The inner catheter can then be advanced selectively into the vertebral artery, optionally over a wire. When desirable to advance the catheter further as well, the controllers for steering may be placed in a neutral position or another desired position and the catheter can be advanced over the inner catheter and/or wire more distally as well.

In another method (technique), the catheter, introduced via a right radial artery, can be advanced into the aortic arch. The catheter can then be steered and moved to engage the origin of the Left Common Carotid Artery. The inner catheter can then be advanced selectively into the Left Internal carotid artery or the Left External Carotid artery, optionally over a wire. When desirable to advance the catheter further as well, the controllers for steering may be placed in a neutral position or another desired position and the catheter can be advanced over the inner catheter and/or wire more distally as well.

In another method (technique), the catheter, introduced via a right radial artery, can be advanced into the aortic arch or the Innominate artery. The catheter can then be steered and moved to engage the origin of a Left Common Carotid Artery with a bovine origin. The inner catheter can then be advanced selectively into the Left Internal carotid artery or the Left External Carotid artery, optionally over a wire. When desirable to advance the catheter further as well, the controllers for steering may be placed in a neutral position or another desired position and the catheter can be advanced over the inner catheter and/or wire more distally as well.

In another method (technique), the catheter, introduced via a right radial artery, can be advanced into the aortic arch or the Innominate artery. The catheter can then be steered and moved to engage the origin of a Right Common Carotid Artery from the Innominate artery. The inner catheter can then be advanced selectively into the Right Internal carotid artery or the Right External Carotid artery, optionally over a wire. When desirable to advance the catheter further as well, the controllers for steering may be placed in a neutral position or another desired position and the catheter can be advanced over the inner catheter and/or optional wire more distally as well.

In another method (technique), the catheter, introduced via a right radial artery, can be advanced into the Right Subclavian Artery. The inner catheter can then be advanced selectively into the Right Internal carotid artery or the Right vertebral Artery, optionally over a wire. When desirable to advance the catheter further as well, the controllers for steering may be placed in a neutral position or another desired position and the catheter can be advanced over the inner catheter and/or wire more distally as well. Alternatively, sometimes the catheter's distal end can be steered directly from the Subclavian artery into the Right Vertebral Artery directly.

Similar techniques in opposite directions may be used to access the precerebral vessels from a left radial artery access site as well. Once a precerebral artery is selected, angiography can be performed. When desired, additional devices can be inserted for interventions as well. More distal superselective access of the head, more distal vasculature in the brain or other circulations may be obtained as well, and interventions can be performed.

In accordance with another aspect of the present invention, a method of inserting a catheter to a target site is provided comprising the steps of:
a) inserting a first wire having a first length into a vessel of a patient via percutaneous access;
b) inserting a distal end of a catheter with a dilator positioned therein over a proximal end of the first wire;
c) advancing the catheter and dilator over the first wire until the proximal end of the first wire is directed by the dilator out of a side hole of the catheter;
d) removing the first wire by pulling it proximally out of the dilator;
e) inserting a second wire through a proximal end of the catheter and dilator and advancing the second wire out the distal end of the catheter, the second wire having a second length longer than a length of the catheter; and
f) advancing the catheter over the second wire to the target site.

In some embodiments, the dilator has a side directing ramp to direct the first wire toward the side hole of the catheter. The side ramp can be aligned with the catheter side hole. In some embodiments, the side hole is covered e.g., has a seal. The method can further include the steps of removing the dilator. The method can include the step of injecting a “radial cocktail.” In some embodiments, the vessel is a radial artery.

In accordance with another aspect of the present invention, a method of inserting a catheter to a target site is provided comprising the steps of:
a) inserting a first wire having a first length into a body of a patient via percutaneous access into a radial artery;
b) inserting over a proximal end of the first wire a distal end of a catheter with a side hole and a dilator with a side directing ramp positioned therein, the ramp directed towards the side hole;
c) advancing the catheter and dilator over the first wire until the proximal end of the first wire is directed by the dilator out of the side hole of the catheter;
d) removing the first wire by pulling it proximally out of the dilator;
e) removing the dilator;
f) inserting a second inner catheter over a second wire through a proximal end of the catheter and advancing the second wire and a distal segment of the second catheter out the distal end of the catheter, the second wire having a second length longer than a first length of the first wire and longer than a length of the catheter, the second catheter having a length longer than the length of the catheter; and
g) advancing the catheter over the second wire and the second catheter to the target site.

The method can include the step of injecting a “radial cocktail.” In some embodiments, the side hole is covered, e.g., has a seal.

In the methods described herein, the catheter inserted can have at least one steering zone. The at least one steering zone can comprise at least two active steering zones and the catheter can further have at least two independent controllers, each controller manipulating an independent steering zone, near the proximal end of the catheter, to a target site. In some embodiments, these methods utilizing a steerable catheter can include one or more of the following steps:
a) withdrawing the catheter and wire proximal to the desired steering zone;
b) adjusting the controller to steer the catheter to a desired shape and position; and/or
c) actively steering the catheter to a desired shape and position via manipulation of the at least one controller near the proximal end of the catheter.

In methods described herein utilizing steerable or non-steerable catheters, the methods can include one or more of the following steps:
a) advancing said second catheter over the second wire to a more distal target site, the target site optionally being a precerebral or cerebral vessel;
b) further advancing the second inner catheter and second wire more distally;
c) further advancing the catheter;
d) removing the second catheter and/or second wire; and/or
e) advancing additional devices (including catheters, wires, interventional devices, etc.) through the catheter to a more distal target site. Examples of a more distal target site include a precerebral or cerebral vessel.

In some embodiments, the dilator of the various systems and methods disclosed herein includes a ramp, and the proximal end of the wire is directed out of the side hole via the ramp, the ramp positioned adjacent the side hole in the catheter. In some embodiments, the cover provides a seal to reduce leakage therethrough, and the first wire is forced against the seal to move the seal to extend outwardly through the side hole. In some embodiments, the seal comprises a flap movable between a closed position to reduce leakage and an open position, the flap movable to the open position by the first wire extending outwardly from a lumen of the catheter.

The methods disclosed herein can further include the steps of removing the dilator and removing the second wire and/or the second catheter. The methods disclosed herein can include the initial step of inserting a needle percutaneously into the artery, then removing the needle while leaving the wire in place.

Among other advantages, the rapid exchange of the present invention substantially eliminates the need for a separate lumen for the rapid exchange wire to pass through, thereby maximizing the diameter of the catheter's primary lumen. This can have many advantages. For example, the velocity of flow is inversely proportional to the radius to the fourth power (Poiseuille's law) so these rapid exchange catheters can have substantially increased rates of flow. This also improves aspiration rates for aspiration catheters.

The methods and devices of the present invention can be used in various procedures. For example, when used for carotid access it can improve ease of percutaneous carotid access in difficult anatomical scenarios, thereby decreasing the risks of these percutaneous approaches. Catheters of the present invention that are optimized for right carotid access via a transfemoral route will typically have a longer segment resting on the lesser arch of the aorta than corresponding catheters that are optimized for left carotid access. Embodiments of the present invention include transfemoral and arm access arch fulcrum catheters. The catheters may optionally have active steerability of their respective bends which can be provided by a wire or wires in the wall of the catheter to provide a series of axially spaced bending zones. An example of such arch fulcrum catheters can be found in U.S. Pat. No. 10,258,371 and pending U.S. application Ser. No. 17/279,210, filed Mar. 24, 2021, (Publication No. 2021/0307892) and U.S. application Ser. No. 17/423,502, filed Jul. 16, 2021, (Publication No. 2022/0118219) the entire contents of which are incorporated herein by reference.

In accordance with another aspect of the present invention, a method of obtaining percutaneous radial artery access for a catheter having two independent steering zones is provided comprising:
a. percutaneously advancing a needle through a skin of a patient overlying a radial artery into the radial artery so the needle tip is in a lumen of the radial artery;
b. advancing a wire through the needle into the radial artery so that a distal end of the wire is in a more proximal radial artery and a proximal end of the wire is outside the patient and outside a proximal end of the needle;
c. removing the needle;
d. advancing a dilator having a distal taper and a lumen, wherein the lumen is centrally located at a distal end of the dilator and courses substantially straight for a distance between about 1 cm to about 40 cm, and then the lumen is directed to a side hole via a ramp, over the wire until the distal end of the dilator is in the radial artery and the proximal end of the wire is projecting out the side hole, the side hole and proximal wire both remaining outside a patient's body;
e. removing the wire;
f. advancing a catheter having at least two independent steering zones over the dilator and thereby positioning a distal end of the catheter into the radial artery (see e.g., FIG. 38); and
g. removing the dilator.

In some embodiments, the step of percutaneously advancing a needle through the skin of a patient advances the needle through both walls of the radial artery before pulling the needle back. In some embodiments, the step of percutaneously advancing a needle through the skin of the patient comprises advancing the needle under ultrasound. In some embodiments, the step of percutaneously advancing a needle through the skin of the patient comprises advancing the needle under radiologic guidance. In some embodiments, the method includes the step of infusing vasodilator drugs to reduce the incidence of clinically significant arterial vasospasm.

In accordance with another aspect of the present invention. a method to catheterize a left vertebral artery arising from a left subclavian artery via a percutaneous right radial artery access is provided comprising
a) utilizing a percutaneous access technique, place a primary catheter having at least two independent steering zones near a distal end of the catheter, the at least two independent steering zones including a proximal steering zone spanning a length between about 30 mm and about 150 mm and a distal end of the proximal steering zone positioned at least about 20 mm away from a distalmost end of the catheter, and wherein the proximal steering zone is configured to deflect up to about 230 degrees with a turning diameter between about 30 mm and about 100 mm, the catheter percutaneously inserted through a skin of a patient overlying a right radial artery so the distal end is in the right radial artery;
b) advancing the distal end of the primary catheter, through the right radial artery, right brachial artery, right axillary artery, right subclavian artery and innominate artery and into an aortic arch of the patient (see e.g., FIG. 39);
c) withdrawing an elongated member positioned in the primary catheter proximal to a midpoint of the proximal steering zone of the catheter;
d) deflecting the proximal steering zone between about 140 to about 230 degrees and steering the distal end of the primary catheter into a proximal left subclavian artery; and
e) advancing a distal end of the elongated member into the left subclavian artery, and subsequently into a left vertebral artery (see e.g., FIG. 38).

In some embodiments, the catheter is inserted under fluoroscopic guidance. In some embodiments, the primary catheter is advanced over a wire. In some embodiments, the primary catheter is advanced over a second catheter. In some embodiments, the elongated member can comprise an inner wire; in other embodiments, the elongated member can comprise an inner secondary catheter.

In some embodiments, the elongated member is withdrawn completely proximal to the proximal steering zone. In some embodiments, the elongated member is withdrawn completely from the primary catheter.

In some embodiments the elongated member is advanced over a wire.

In accordance with another aspect of the present invention a method to catheterize a left internal carotid artery arising from a left common carotid artery arising from an aortic arch via a percutaneous right radial artery access is provided comprising:
a. utilizing a percutaneous access technique, place a tertiary catheter having at least two independent steering zones near a distal end of the catheter, wherein the at least two steering zones include a distal steering zone and a proximal steering zone, the distal steering zone spanning a length between about 20 mm and about 90 mm, with the distal end of the distal steering zone positioned between 0 to about 20 mm away from a distalmost end of the catheter, and wherein the distal steering zone is configured to deflect up to about 270 degrees with a turning diameter between about 15 mm and about 60 mm, the catheter percutaneously inserted through the skin overlying a right radial artery, so the distal end is in the right radial artery;
b. advancing the distal end of the tertiary catheter over an elongated member, through the right radial artery, right brachial artery, right axillary artery, right subclavian artery and innominate artery and into an aorta arch of the patient (see e.g., FIG. 40);
c. withdrawing the elongated member proximal to a midpoint of the proximal steering zone;
d. deflecting the distal steering zone between about 140 and about 200 degrees and steering the distal end of the tertiary catheter into a proximal left common carotid artery (see e.g., FIG. 40); and
e. advancing the distal end of the elongated member into the left common carotid artery and subsequently into a left internal carotid artery.

In some embodiments, the catheter is inserted under fluoroscopic guidance. In some embodiments, the primary catheter is advanced over a wire. In some embodiments, the primary catheter is advanced over a second catheter. In some embodiments, the elongated member can comprise an inner wire; in other embodiments, the elongated member can comprise an inner secondary catheter.

In some embodiments, the elongated member is withdrawn completely proximal to the proximal steering zone. In some embodiments, the elongated member is withdrawn completely from the primary catheter.

In some embodiments the elongated member is advanced over a wire.

Devices described herein are designed in some embodiments to be particularly useful for use via a unilateral percutaneous radial artery or other arm vessel access to access, image, and treat lesions in the precerebral and cerebral vasculature. Notwithstanding this, the devices and methods described herein will also facilitate procedures in other parts of the body, via access through a vessel in the arm and/or elsewhere, and can also facilitate precerebral and cerebral vessel access, imaging and treatment via other access sites.

Devices described herein can also have uses in procedures done transvenously, as well as via other access sites.

### BRIEF DESCRIPTION OF THE DRAWINGS

Preferred embodiments of the present invention are described herein with reference to the drawings wherein:

FIG. 1A is a plan view showing radial access with a short wire in accordance with one embodiment of the method of the present invention;

FIG. 1B is a plan view showing femoral access with a short wire in accordance with another embodiment of the method of the present invention;

FIG. 2A is a plan view showing the catheter and dilator inserted over the proximal end of the short wire in accordance with the radial access method of FIG. 1A;

FIG. 2B is a plan view showing the catheter and dilator inserted over the proximal end of the short wire in accordance with the femoral access method of FIG. 1B;

FIG. 3 is a cross-sectional view showing the catheter and dilator of FIG. 2A inserted over the proximal end of the short wire;

FIG. 4A is a cross-sectional view similar to FIG. 3 showing the short wire extending out of the side hole (rapid exchange port) of the catheter;

FIG. 4B is a cross-sectional view similar to FIG. 4A showing the short wire being withdrawn from the dilator and catheter;

FIG. 5 is a cross-sectional view similar to FIG. 4 showing the short wire fully removed from the catheter;

FIG. 6 is a cross-sectional view similar to FIG. 5 showing a guidewire inserted though the lumen of the dilator;

FIG. 7 is a cross-sectional view similar to FIG. 6 showing the catheter and dilator advanced over the guidewire where the dilator extends past the distal end of the guidewire;

FIG. 8 is a cross-sectional view similar to FIG. 7 showing the guidewire removed from the dilator and catheter;

FIG. 9 is a cross-sectional view similar to FIG. 8 showing the dilator removed from the catheter;

FIG. 10A is a cross-sectional view similar to FIG. 9 showing a device inserted through the lumen of the catheter;

FIG. 10B is a cross-sectional view similar to FIG. 10A showing a wire extending through the device;

FIG. 10C is a cross-sectional view similar to FIG. 10A showing the wire and device partially withdrawn to enable steering of the catheter;

FIG. 11 is a side perspective view of one embodiment of the catheter of the present invention having a tube coupling two segments;

FIG. 12 a side view of the catheter of FIG. 11 showing the side port (hole) covered by an elastic material and the short wire extending through the side hole;

FIG. 13 is a side perspective view of an alternate embodiment of the catheter of the present invention having an internal tube;

FIG. 14 is a cross-sectional view of the catheter of FIG. 13 showing the short wire extending through the side port (hole) of the catheter;

FIG. 15 is a side perspective view of an alternate embodiment of the catheter of the present invention having a mechanical seal for the side port;

FIG. 16 a side view of the catheter of FIG. 15 showing the short wire extending through the side port of the catheter;

FIG. 17 is a side perspective view of an alternate embodiment of the catheter of the present invention having a flap to seal the side port;

FIG. 18 a side view of the catheter of FIG. 17 showing the short wire extending through the side port of the catheter;

FIG. 19 is a side perspective view of an alternate embodiment of the catheter of the present invention having a flap formed in the inner liner of the catheter to seal the side port;

FIG. 20 a side perspective view of the catheter of FIG. 19 showing the short wire extending through the side port of the catheter;

FIG. 21 a side perspective view of an alternate embodiment of the catheter of the present invention having a flap attached to a ring;

FIG. 22 is a side perspective view of the catheter of FIG. 21 showing the short wire extending through the side port of the catheter;

FIG. 23 is a side perspective of one embodiment of the catheter of the present invention having a proximal luer lock, a handle with a proximal wheel that controls a proximal steering zone, and a distal wheel that controls a distal steering zone;

FIG. 24 is a side of a catheter of the present invention having a distal and proximal deflection (steering) zone;

FIG. 25 is a side view showing deflection of the catheter of FIG. 24;

FIG. 26 is a transverse cross-sectional view of the catheter of FIG. 24;

FIG. 27 is a transverse cross-sectional view of an alternate embodiment of the catheter of the present invention;

FIG. 28 is a transverse cross-sectional view of an alternate embodiment of the catheter of the present invention;

FIG. 29 is a transverse cross-sectional view of an alternate embodiment of the catheter of the present invention;

FIG. 30 is a transverse cross-sectional view showing a pull ring positioned within the wall of the catheter (the pull wires are not shown) in accordance with an embodiment of the present invention;

FIG. 31 is a transverse cross-sectional view showing an alternate embodiment of the pull ring of the present invention;

FIG. 32 a side perspective view of a distal region of an embodiment of a catheter of the present invention showing the proximal pull ring;

FIG. 33 is a transverse cross-sectional view of the catheter of FIG. 32;

FIG. 34 is a cross-sectional view taken along line A-A of FIG. 33;

FIG. 35 is a side view of an alternate embodiment of the dilator of the present invention;

FIG. 36 is a side view of an alternate embodiment of the dilator of the present invention;

FIG. 37 is a side view of an alternate embodiment of the dilator of the present invention.

FIG. 38 illustrates insertion of a steerable catheter of the present invention into the radial artery over a dilator;

FIG. 39 illustrates insertion of a steerable catheter of the present invention into the left vertebral artery; and

FIG. 40 illustrates insertion of a steerable catheter of the present invention into the left internal carotid artery.

### DETAILED DESCRIPTION OF THE INVENTION

Referring now to the drawings wherein like reference numerals identify similar structures, element, and features, various embodiments of the presently disclosed systems, devices and methods will be discussed.

Note as used herein, the term “proximal” denotes a region or portion of the catheter, dilator, instrument, etc. closer to the user and the term “distal” denotes a region or portion further from the user.

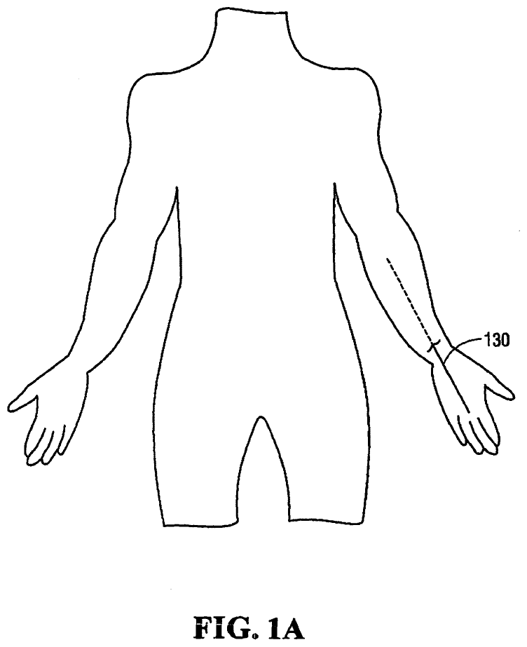

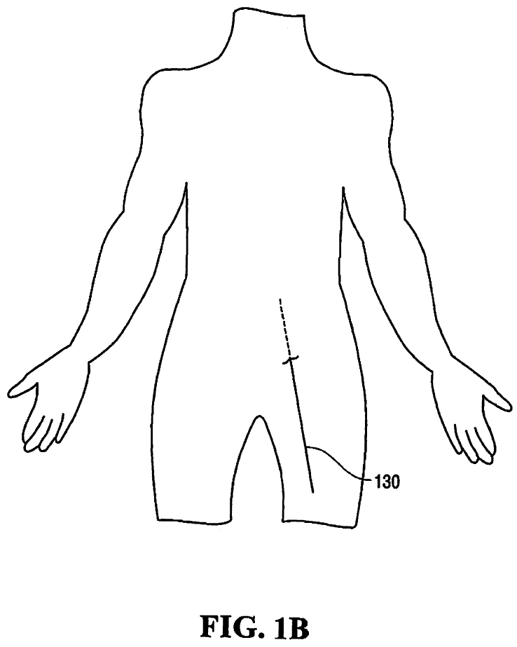

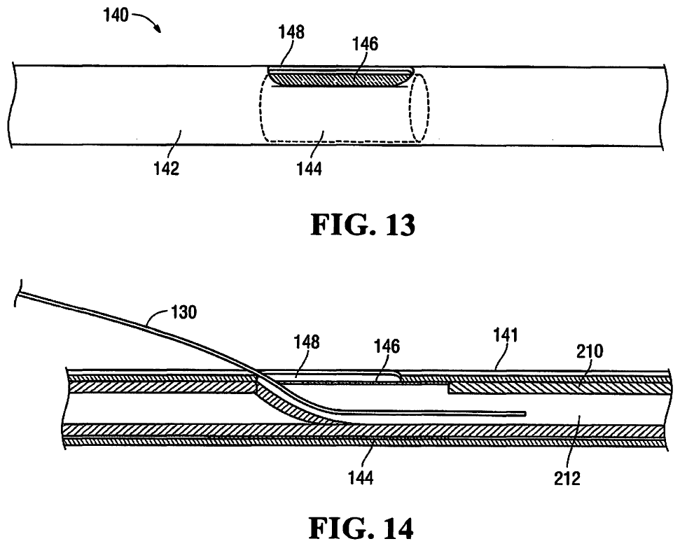

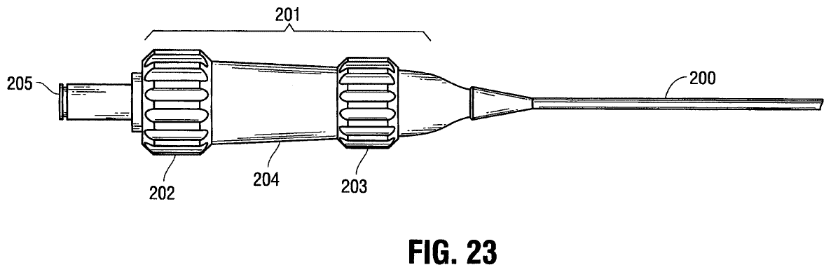

FIGS. 1-12 show methods of use of the rapid exchange seal of the present invention; FIGS. 13-22 show various embodiments of the rapid exchange seal of the present invention; and FIGS. 23-34 shows various embodiments of a steerable (deflectable) catheter of the present invention.

The present invention provides in some embodiments a rapid exchange port, formed as a side opening in a wall of the catheter, with a seal to prevent, or at least reduce, egress of fluids. The seal can be displaced by a wire extending through the port. The side opening is typically within about 10 cm to about 20 cm of the distal end hole of the catheter, although it can be at other locations.

The present invention also in some embodiments provides a system which includes a) a catheter with a rapid exchange port and b) a dilator with a wire directing structure to redirect the wire through the rapid exchange port and out of the catheter. The rapid exchange port preferably includes a seal which is moved out of the way, i.e., to an open position, by the wire as the dilator is advanced over the wire. Without the wire, the seal is preferably in a closed position to reduce leakage out of the port.

In the illustrated embodiments, the wire directing structure includes an internal ramp in the dilator. The dilator is positioned within the rapid exchange catheter so the ramp is aligned with the rapid exchange port and as the dilator (with the externally concentrically positioned catheter) is advanced over the short wire, the short wire is redirected out of the dilator by the ramp to slightly displace the seal so it can exit past the seal and exit the port of the catheter. This is described in more detail below. One advantage of the present invention is in certain instances it can avoid having to use a separate introducer sheath to gain initial access to the vasculature. This will be appreciated by the detailed discussion below of the method of use.

Among other advantages, the rapid exchange catheters of the present invention substantially eliminate the need for a separate lumen for the rapid exchange wire to pass through, thereby maximizing the diameter of the catheter's primary lumen. This can have many advantages. For example, the velocity of flow is inversely proportional to the radius to the fourth power (Poiseuille's law) so these rapid exchange catheters can have substantially increased rates of flow. This also improves aspiration rates for aspiration catheters.

The rapid exchange port of the present invention can be provided on a flexible catheter. It can also be provided on a steerable catheter, wherein the catheter has several steerable zones along a longitudinal axis such as steerable zones disclosed in pending U.S. application Ser. No. 17/279,210, filed Mar. 24, 2021, (Publication No. 2021/0307892) which discloses catheters with multiple passive or active bends bendable at various angles and in U.S. application Ser. No. 17/210,778, filed Mar. 24, 2021, (Publication No. 2021/0236257) which discloses catheters which include a plurality of segments and a plurality of pull wires which can be built into the wall of the catheter. The catheters disclosed in the U.S. application Ser. No. 17/210,778 include a plurality of inactive (passive) segments and a plurality of active (steerable, deflectable, articulable) segments defining bends that are connected to the plurality of pull wires at connection points and spaced along a longitudinal axis of the catheter. The inactive segments and the active segments are arranged in a staggered pattern such that the catheter alternates between inactive segments and active segments. Each active segment is connected to a corresponding (single) pull wire such that the number of pull wires corresponds to the number of active segments. Upon the application of an axial (pulling) force to each of the pull wires, the corresponding active segment is deflected (articulated) to thereby reconfigure (actively steer) the catheter between a first (initial, normal) configuration in which the catheter includes a (generally) linear configuration, and a second (subsequent, deflected) configuration in which the catheter includes a non-linear configuration. The bends may lie substantially within the range of approximately 0 degrees to approximately 270 degrees. The catheters having the rapid exchange port can alternatively include a push wire wherein pushing (rather than pulling) effects deflection or can alternatively have a neutral position wherein pushing or pulling effects deflection in opposing directions. The catheters having the rapid exchange port can also include rotational wires for rotating the catheter as in pending U.S. application Ser. No. 17/214,021, filed Mar. 26, 2021 (Publication No. 2021/0259860).

The entire contents of U.S. application Ser. Nos. 17/279,210, 17/210,778 and 17/214,021 are incorporated herein by reference and the steerable/bend zones, pull wires, connection points, etc. are fully applicable to the catheters disclosed herein.

The rapid exchange ports of the present invention can also be used with a steerable (deflectable) catheter disclosed herein.

FIG. 23 illustrates an embodiment of a steerable catheter of the present invention. Catheter 200 has a handle 201 attached at the proximal end. A proximal wheel 202 controls a proximal steerable segment (proximal steering zone), and a distal wheel 203 controls a separate (independent) distal steering segment (distal steering zone). The catheter includes at least one gear within casing 204, and at least one wire for each steering zone extending substantially within the wall of catheter 200, extending from a proximal attachment controlled by an independent gear, and each independent gear is controlled by an independent wheel (e.g., wheel 202 or wheel 203). Thus, each wire is operatively connected to a gear and controls steering of a steerable segment via shortening or lengthening of the wire via the respective wheel rotating the gear in the desired direction to pull or push a given wire. Catheter 200 also can have a luer lock 205 at its proximal end. Catheter 200 may also include at least one rapid exchange side hole (not shown), which may be partially or fully resealable via a scaling port as disclosed herein.

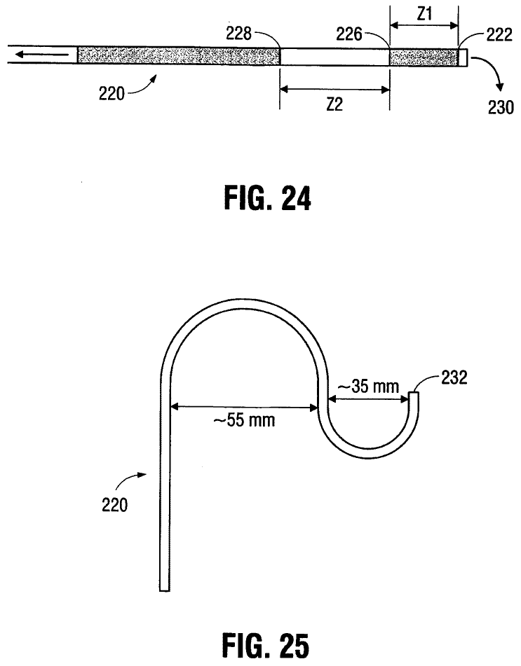

FIG. 24 illustrates an alternate embodiment of the catheter of the present invention having two independent steering zones. The distal deflection (steering) zone Z1 has a distalmost end 222 and a proximalmost end 224 and the proximal deflection (steering) zone Z2, which is proximal of zone Z1, has a proximalmost end 228 and distalmost end 226 (at the proximalmost end 224 of distal zone Z1). Each zone Z1, Z2 is independently deflectable/steerable by a wheel, lever or other mechanism. The distal tip of the catheter 220 is designated by reference numeral 230.

The deflection zones Z1, Z2 can have various lengths. In one example, the distal deflection zone Z1 can have a length of between about 20 mm and about 90 mm and more specifically a length of about 50 mm and the proximal deflection zone Z2 can have a length of about 30 mm to about 150 mm and more specifically about 75 mm (75 mm from the location of the most distal pull ring). In preferred embodiments, the proximal deflection zone Z2 is longer than the distal deflection zone Z1 so the proximal turning diameter is larger than the distal turning diameter.

The proximal deflection zone Z2 preferably ends between about 5 mm and about 95 mm away from the distalmost end 230 of catheter 220, and more preferably ends at least 20 mm away from the distalmost end 230. The distal zone Z1 preferably ends at least 20 mm from the distalmost end 230 of catheter 220 or alternatively can end at the distalmost tip.

Note that the foregoing dimensions are provided by way of example as other dimensions for the distal and proximal zones, and other distances from the distal tip of the catheter, are also contemplated. Note in some embodiments the proximal zone Z2 can overlap partially with the distal deflection zone Z1, however, in preferred embodiments, they do not overlap.

FIG. 25 illustrates an embodiment in which the distal zone can deflect in two opposite directions, e.g., up or down from the orientation of FIG. 24. The deflection radii as shown show approximately 55 mm on the proximal deflection and about 35 mm on the distal deflection. As can be appreciated, the proximal deflection zone deflects in a first direction, e.g., toward the right, and the distal deflection zone deflects distally, thus forming the configuration of FIG. 25. As can be appreciated, this provides one example of the deflected catheter configuration that can be achieved with the two deflection zones as other configurations are also contemplated. Note in FIG. 25, proximal deflection is about 180 degrees, however, in preferred embodiments, proximal deflection can be up to 230 degrees. Distal deflection can be up to 270 degrees. Other degrees of deflection are also contemplated.

In some embodiments, the direction of steer of the two zones is in substantially opposite directions. In other preferred embodiments, the direction of steer in the two zones is in the same direction, e.g., substantially +/−40 degrees, with the pull wires (or alternatively the push wires) of the two deflection zones substantially side-by-side in the wall of the majority of the length of the catheter from the distal end of the proximal deflection zone and proximally. In an “enhanced function” embodiment seen in FIG. 25, the distal deflection zone can also deflect in a direction substantially opposite the direction of the primary direction of deflection of the proximal deflection zone so the distal deflection zone in the “enhanced function” catheter can deflect in two distinct directions, which are substantially opposite to each other. In the preferred embodiment, this is accomplished by another wire (a “secondary wire” which can be in the form of a pull or push wire) in addition to the “primary wire” which is also positioned in the wall of the catheter and located on the opposite side of the distal deflection zone “primary” wire (preferably 180 degrees in cross-section; but could be +/−40 degrees).

In preferred embodiments, all wires effect deflection by attaching on the distal end of a “deflection zone” and having a “pulling mechanism” in a handle that pulls on the wire. The proximal end of a deflection zone is dictated by changes in the wall composition (stiffness and other) between the deflection zone and the adjacent regions of the catheter proximal to the deflection zone.

In preferred embodiments, each wire is pulled to effect deflection. Pulling of the wire will effect deflection initially in the direction of the wire when the wire is pulled (or shortened). However, in alternate embodiments, each wire can also be pushed to effect an opposite deflection, i.e., deflect in a direction initially opposite the wire. In such embodiments, the handle will have a mechanism to push the wire.

In the preferred embodiment, an opposite pulling wire is provided as well to deflect the distal deflection zone in the opposite direction when desired.

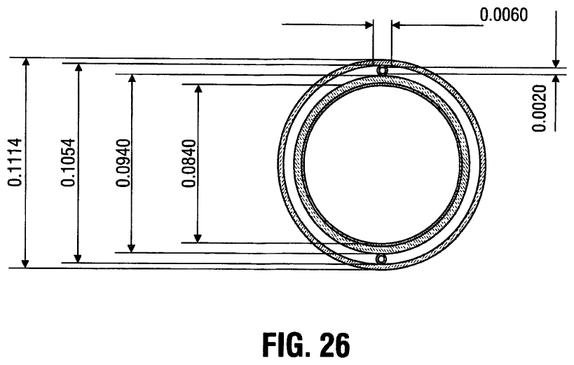

Pull wires or various dimensions can be utilized. In one embodiment by way of example the pull wire has an oval configuration of the dimensions about 0.002″ x about 0.004″ (inches) or about 0.002″ x about 0.006″ (inches), with the wires embedded in the wall of the catheter as shown in FIG. 26 in a catheter having an outer diameter (outer wall) of about. 0.1114 inches and an inner wall diameter of about 0.0940″ to form a lumen of about 0.0840 inches. Note the dimensions are provided by way of example as other wire and catheter wall/lumen dimensions are also contemplated.

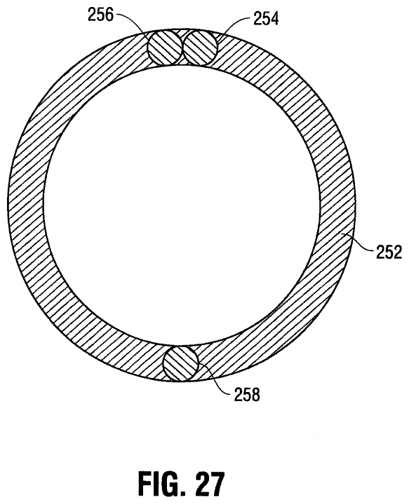

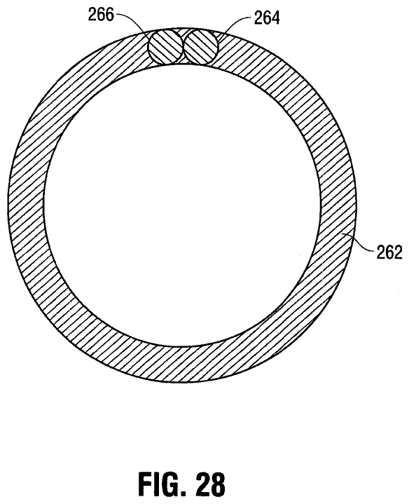

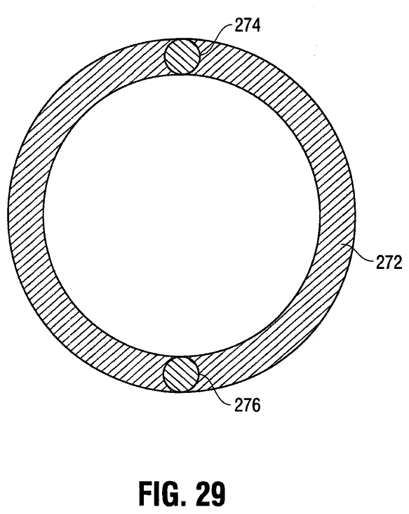

FIGS. 27-29 illustrate transverse cross-sectional views of various embodiments of the steering mechanism of the present invention. In FIG. 27, the transverse cross-sectional view taken through a segment of the catheter between the handle and the proximal pull ring shows two pull wires 254, 256 positioned substantially in the wall 252 of the catheter and substantially side-by-side, which act independently to deflect two different deflection zones in a substantially similar direction of initial deflection. One of the wires, e.g., wire 254, would extend further than the other wire 256 to connect with the distal ring for deflection of the distal zone. The wire 256 would connect with the proximal pull ring. Also provided is a single wire 258 positioned substantially in the wall of the catheter on the opposite side, which inserts into the distal pull ring, and when pulled deflects the distal deflection zone in a direction initially substantially opposite the direction of initial deflection of the wire 254 (and wire 256). Thus, in this embodiment, the distal deflection zone is bidirectional due to the two oppositely positioned wires and the proximal deflection zone is unidirectional due to the single wire. In the embodiment of FIG. 28, showing a transverse cross-section taken between the handle and the proximal pull ring, two pull wires 264, 266 are positioned substantially in the wall 262 of the catheter substantially side-by-side, which act independently to deflect two different deflection zones in a substantially similar direction of initial deflection. In the embodiment of FIG. 29, showing a transverse cross-section taken between the proximal pull ring and the distal pull ring, both wires 274, 276 are positioned substantially in the wall 272 of the catheter on opposing sides, and are inserted into/are attached to the distal pull ring. When each wire is 274, 276 is pulled, it creates deflection in the distal deflection zone, each in substantially opposite directions on initial deflection relative to each other.

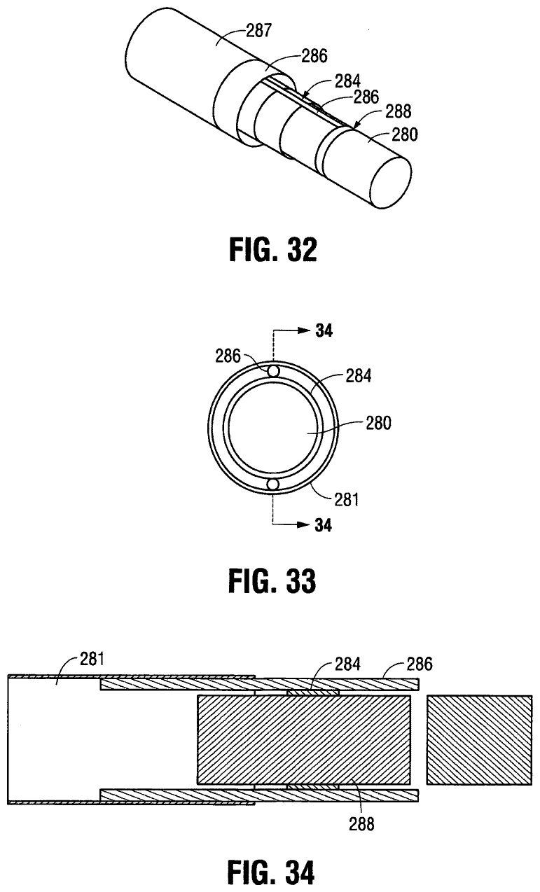

The wire can be attached to the pull ring as shown for example in FIGS. 32-34. Catheter 280 has pull wire 286 attached to proximal pull ring 284. It can be attached to the outside or inserted into and/or through the ring for attachment. The shaft includes an outer Pebax (or other materials) jacket 281, a braid 282 and an inner liner 288. As shown, the pull wire 286 is offset from a longitudinal axis of the catheter and can extend distally past the pull ring 284. Each deflection zone has a separate independent pull ring at the distal end of the deflection zone. The pull wire attaches to the pull ring to effect deflection.

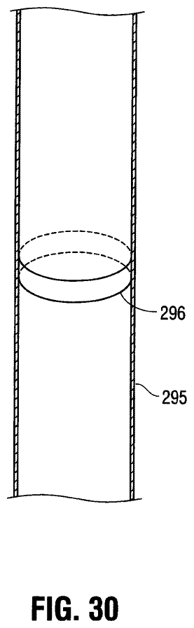

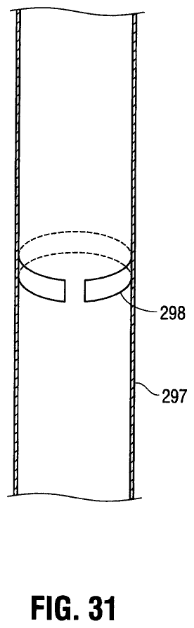

The pull ring can be embedded in the wall of the catheter such as pull ring 296 of catheter 295 of FIG. 30. (The pull wires are not shown in FIG. 30). The pull ring can be a closed shape, e.g., circular shaped, as shown in FIG. 30 or alternatively can be an open shape such that it has a gap as in pull ring 297 of catheter 298 of FIG. 31. (The pull wires are not shown in FIG. 31). The open shape can provide a pull ring of various shapes such as the C or U-shape of FIG. 31. The use of a pull ring with a gap enables the opposite side wire attached to the distal deflection ring to traverse the proximal ring through the gap instead of inside or outside the ring, and thereby the wall thickness of this segment at the proximal pull ring can be minimized, allowing maximizing internal diameter for a given outer diameter of the catheter i.e., allowing egress of the wire in the wall of the catheter while limiting the total thickness of the catheter wall.

The catheter can have a single handle “in-line” and surrounding a proximal portion of catheter, with the proximal end of the catheter ending proximal to the handle as shown for example in FIG. 23. In another embodiment, the handle(s) is on a “branch” to the side (i.e., forming part of a “Y” near the proximal end of the catheter).

Preferably, each deflection zone has its own independent steering mechanism. The mechanism can be a “wheel-like” device, a lever, or other mechanisms for controlling the steering wires.

In some embodiments, the handle has spiral grooves and/or pulleys or other mechanisms to allow a longer distance of wire to traverse a given length of handle, so a shorter handle can pull a longer length of wire.

In some embodiments the wire(s) is attached, directly or indirectly, to a threaded gear within the handle that moves the wire forward or back when the steering mechanism is activated.

The steering mechanism in some embodiments is capable of “locking” in place in a given position of deflection. The locking can be automatic via an automatic mechanism such as tension on the movement mechanism, position of the threaded gear, a ratchet, etc. Alternatively, the locking can require an active locking initiated by the user.

As discussed above, instead of a pull wire, a push wire could be utilized to effect deflection. In other embodiments, the wire can have a neutral position where it is pushed for deflection in one direction and pulled for deflection in the opposite direction. Further, rods or other elongated members can be used instead of wires.

The steerable catheters disclosed herein can include a distal side hole, e.g., within about 30 cm of the distal end of catheter and preferably between about 5 cm and about 15 cm. The side hole can have an “auto-scaling” covering or other type of sealing feature as disclosed in detail below, that allows a wire to go through, but is substantially impermeable to fluid (a small leak rate is allowed in some embodiments). The steerable catheters disclosed herein can be used with the dilator disclosed herein.

The rapid exchange port of the present invention in preferred embodiments is optionally self-sealing so that its normal position is closed to reduce or fully prevent leakage, although in alternate embodiments it can be selectively, e.g., manually or other means, opened and/or closed. The sealed port advantageously facilitates injection of fluid since it substantially prevents fluid from exiting through the side port if the seal wasn't present. It also facilitates insertion of instruments through the catheter since it reduces the likelihood of the instrument going through the side port during advancement through the lumen of the catheter.

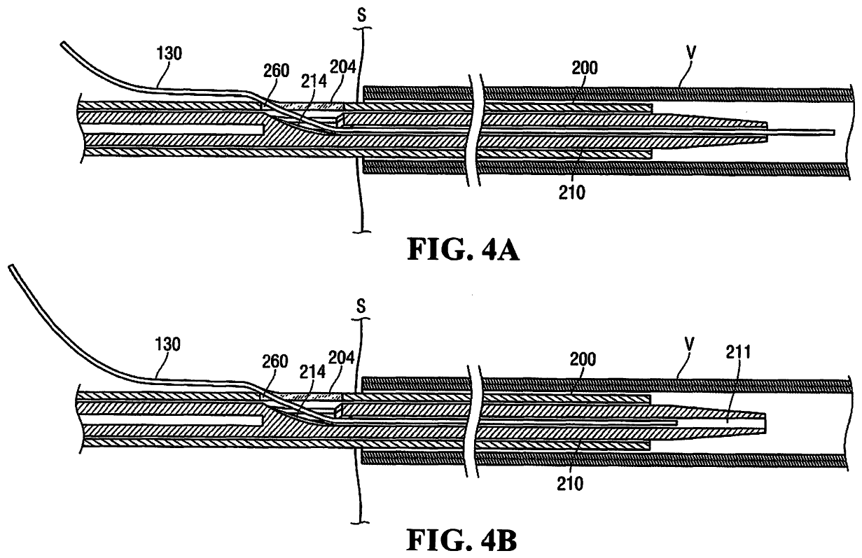

Before turning to details of the method of insertion, various embodiments of the rapid exchange seal will first be discussed. Note an internal dilator is provided within the catheter, as shown for example in FIG. 4A. As noted above, the internal dilator has a wire directing structure, e.g., a ramp, which redirects the wire toward the rapid exchange port and seal. This ramping of the wire is described in more detail below in conjunction with the method of insertion but it should be appreciated at the outset of the discussion of the various seal structures of the present invention that the proximal end of the short wire contacts the ramp and is then forced away from the longitudinal axis toward the side port and pushes the seal out of the way so it can exit the side port as shown for example in FIG. 4A.

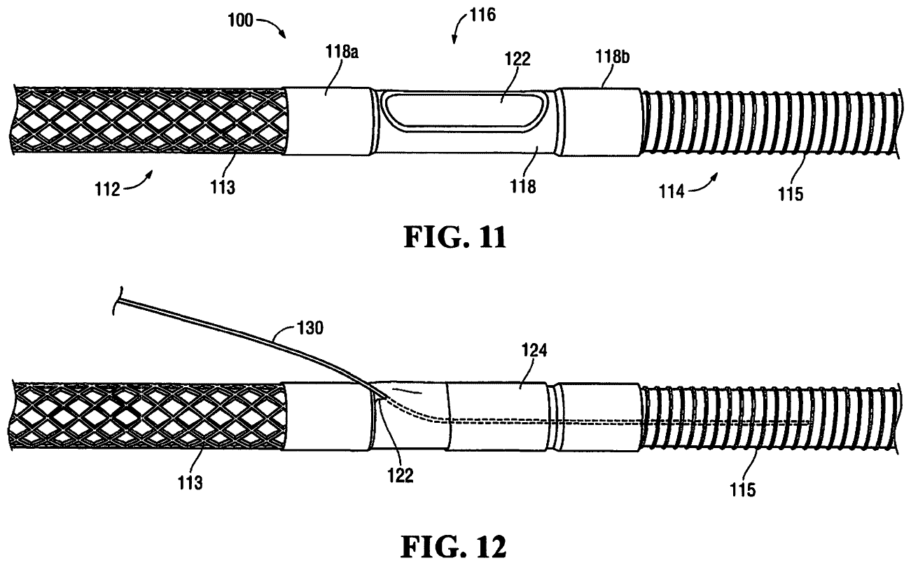

Turning now to the various seal structures, and first to the embodiment of FIGS. 11 and 12, catheter 100 has a proximal end 112, a distal end 114 and a rapid exchange region 116. In the illustrated embodiment, the proximal end 112 is composed of a braid 113 and the distal end 114 is composed of a coil 115. The coil 115 provides more flexibility in the distal region to facilitate navigation through the tortuous vasculature while the braid provides more stiffness to aid insertion/advancement through the vasculature. The coil can have variable pitch and provide various bending zones for steerability. The rapid exchange region 116 includes a tube 118 having an elongated side opening or side port 122 in the side wall. Note the terms “side port,” “side opening” and “side hole” are used interchangeably herein to denote the opening in the side wall of the various catheters disclosed herein.

The tube 118 connects the braid 113 and coil 115 and a proximal end 118a of the tube 118 is placed over the outer diameter of the braid 112 and a distal end 118b of the tube 118 is placed over the outer diameter of the coil 115 as shown. An elastic sleeve 124 is placed over the tube 118, covering the side opening 122 to form a seal. Short wire 130 exits the port 122 as it forces a portion of elastic sleeve 124 out of the way as the wire 130 is directed outwardly from the longitudinal axis of the catheter 100 and out the opening 122 by the guiding (also referred to herein as the redirecting or deflecting) surface, e.g., the internal ramp, of the internal dilator in the manner described in detail below. Such movement opens the seal from its closed position to allow exit of the short wire 130.

In the alternate embodiment of FIGS. 13 and 14, catheter 140 has a short tube 144 within a lumen of the catheter. The tube 144 is positioned adjacent the cutout (opening) 148 in the side wall of the catheter 140 which provides the rapid exchange side port. The tube 144 can have a coil embedded within the wall. The tube 144 has a side opening aligned with the side port of the catheter 140. The tube 144 can include an elastic membrane 146 positioned over the opening to form a seal. As shown in FIG. 14, the short wire 130 extends out the opening 148 (due to the redirecting surface of the dilator) as it moves the membrane 146 of the tube 144 out of the way to open the seal from its closed position. By placing the tube within the internal diameter of the catheter, the outer diameter of the catheter 140 is not increased. The tube in alternate embodiments can form a connector like tube 118 of FIG. 12 to connect two catheter portions.

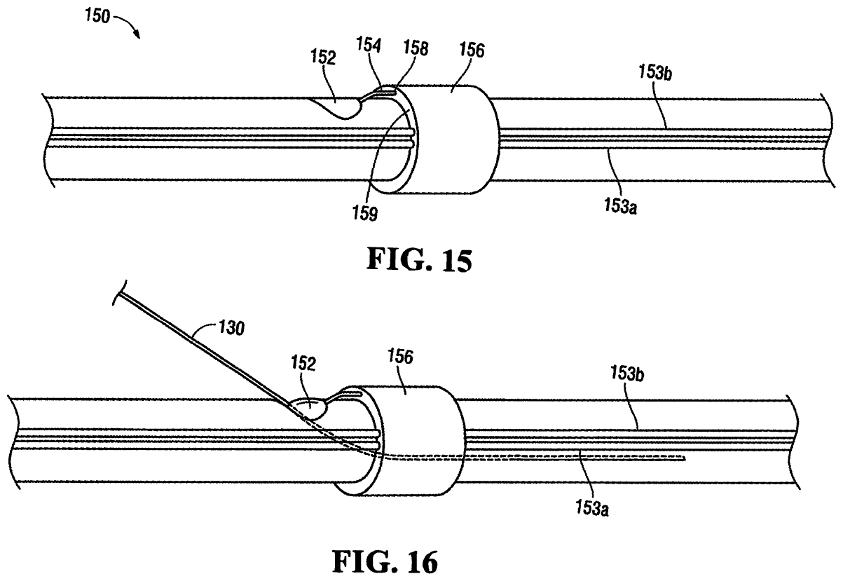

In alternate embodiments of the present invention, a mechanical flap is provided over the rapid exchange port of the catheter that can move between a sealing position and a more open position. An example of a mechanical flap is shown in catheter 150 of FIGS. 15 and 16 wherein the valve (seal) 152 has a connector 154 to connect it to the outer sleeve 156. As shown, the connector can be in the form of a thin rod that at one end extends proximally from valve (seal) 152 and at the other end extends through an opening 158 in wall 159 of sleeve 156. Short wire 130 is shown in FIG. 16 extending past the valve 152 to exit the rapid exchange port of catheter 150 as it moves the flap 152 out of the way, i.e., moves the flap to an open position. The short wire 130 is directed out of the rapid exchange port via the redirecting structure of the dilator positioned within the catheter in the manner described herein. Sleeve 156 may optionally be welded or otherwise connected in-line to the proximal and distal catheter segments to avoid any increase in diameters at the sleeve. Catheter 150, like the other catheters disclosed herein, can be steerable/bendable and can include longitudinally extending lumens 153a, 153b to receive elongated steering members, e.g., wires, rods, etc. The lumens 153a, 153b extend along a length of the catheter 150 and can extend through the sleeve 156 as shown. Alternatively, the lumen can be formed in a catheter 150 such that the steering members can be embedded within the wall of the catheter or substantially embedded in a wall of the catheter 150 and not be in formed lumens as in the embodiment of FIG. 27 for example.

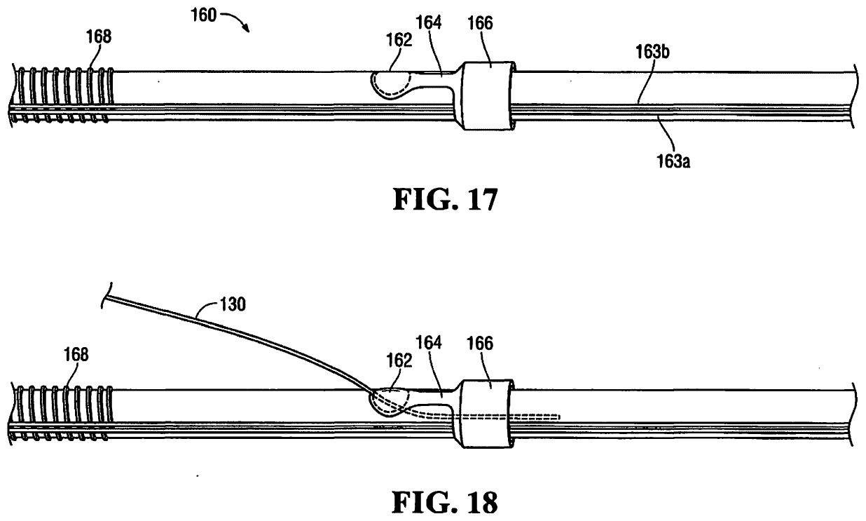

An alternate embodiment of the mechanical flap is shown in the embodiments of FIGS. 17 and 18. The mechanical flap portion 162 covers the rapid exchange port of the catheter 160. In some embodiments, in manufacture, the flap portion can be press fit into the side opening (port) of the catheter 160. The flap portion 162 is formed as one piece with sleeve portion (tube portion) 166 and the sleeve portion 166 is joined to flap 162 via integral connecting portion 164. The tube portion 166 can be formed of Nitinol or other materials. A coil 168 can be provided at the proximal end of the catheter 160. Lumens 163a, 163b extend along the catheter to receive steering rods wires, etc. Alternatively, the steering mechanism can be positioned or substantially positioned within the catheter wall without separate lumens as in FIG. 27 for example. That is, the lumens 163a, 163b can extend along a length of the catheter 150 and can be formed in a wall of the catheter such that the steering members are embedded in or substantially embedded in a wall of the catheter 160. The short wire 130 is shown extending past the valve (seal) 162 in FIG. 18 to open the valve as it pushes past the valve as it engages the redirecting structure, e.g., ramp, of the dilator positioned within the lumen of the catheter 160 in the manner described herein.

Any connector portion in the embodiments described herein can in some embodiments be welded or otherwise connected in-line to the proximal and distal catheter segments to avoid any increase in diameters at the connector.

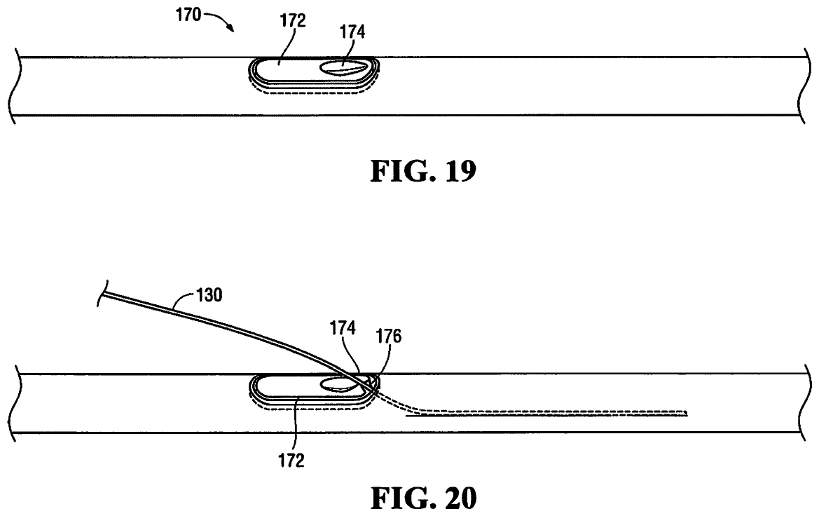

In the embodiment of FIGS. 19-20, the catheter 170 has an inner liner made of materials such as PTFE which acts as a flap. As shown, inner liner 172 provides a seal and is positioned inside the catheter 170 adjacent the side port formed in the wall of the catheter 170. The liner 172 is movable out of the way to an open position by the short wire 130 so the short wire 130 can exit through gap 176. A slit 174 in the liner 172 or in another material placed within, inside or over the liner can provide an alternate exit for the wire 130.

The wire 130 in FIG. 20 is shown exiting at a distal portion of the inner liner 172; however, it should be appreciated, that the wire 130 can exit at a proximal portion of the side port at a proximal region of the liner 172 in a similar manner as the wire exits in the embodiments of FIGS. 4, 11-18 and 21-22 or exit at other regions. It should be appreciated that in the embodiments of FIGS. 4, 11-18 and 21-22, the short wire can alternatively exit through a distal end of the seal as in the embodiment of FIG. 20 or exit at other regions. The region of the seal through which the short wire 130 exits will depend on the alignment of the ramp of the internal dilator with the side port of the catheter as the ramp directs the wire toward the seal and side port.

In alternate embodiments, the liner can be made of PTFE and a stent like structure can be placed over or under the liner, or within, proximal to the side opening, distal to the side opening and/or in the region of the side opening. The stent can modify the stiffness profile of the shaft. A variable pitch braid or multiple braids can be placed over the liner, under the liner and/or embedded in the wall of the liner to control the bend radius.

It should be appreciated that FIGS. 11-22 show examples of seals, but other types of seals and configurations for the catheter ports, e.g., rapid exchange ports, can also be utilized.

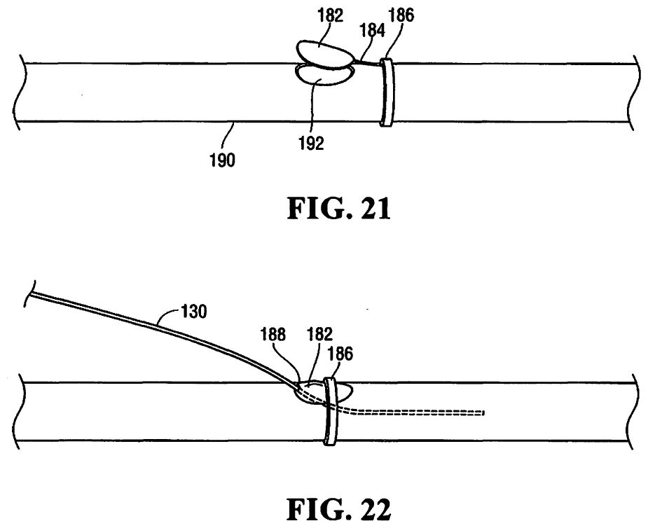

In an alternate embodiment, a seal is provided which can be attached to a catheter. A catheter can have a hole punched or otherwise formed in the side wall, and a mechanical seal such has mini valve in the form of flap 182 of FIG. 21 can be attached to the catheter placed over the side hole 192. As shown in FIG. 21, flap 182 is connected to ring 186 by connector 184. In the preferred embodiments the ring is flush with the remainder of the catheter surface. Ring 186 can in manufacture be positioned around the catheter 190 and slid axially so flap 182 covers the side hole 192. Note the hole can be formed by a tool external of the catheter or alternatively by a tool punching a hole from the inside, a laser, or other means, and such hole can be formed in conventional catheters. The ring 186 in some embodiments can form a radiopaque marker band for imaging. It can be swaged or attached by other methods to the diameter of the catheter.

The various seals of the present invention could either fully seal to prevent any egress of fluids or substantially seal to prevent significant egress of fluids. Partial seals are also envisioned. Such fluids can include for example contrast, therapies, medications, saline and/or other desired fluids.

Note the various steerable mechanisms disclosed above can be utilized with the sealed rapid exchange ports of FIGS. 11-22. Also note that the sealable rapid exchange ports of FIGS. 11-22 can be used without steerable features. The various steerable catheters disclosed herein can have the scalable rapid exchange ports or alternatively be utilized without such sealable rapid exchange ports.

Turning now to the method of use, and with reference to FIGS. 1-10, insertion of the system (device and dilator) of the present invention will now be described. It should be appreciated that any of the catheters described herein can be utilized in the manner described herein. FIGS. 1-10 show a seal in the form of an elastic membrane covering the side port as an example to illustrate the interaction of the components. Thus, it should be understood that the description below of the method of use, and its alternatives, are fully applicable to any of the catheter embodiments discussed above or illustrated in the drawings.

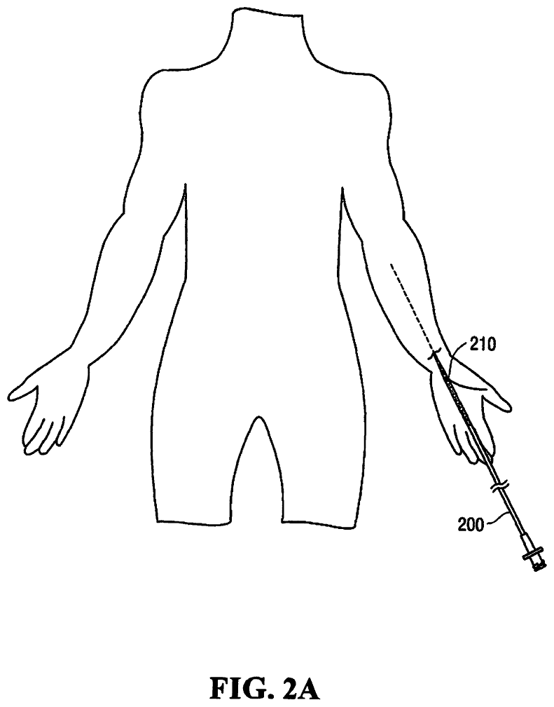

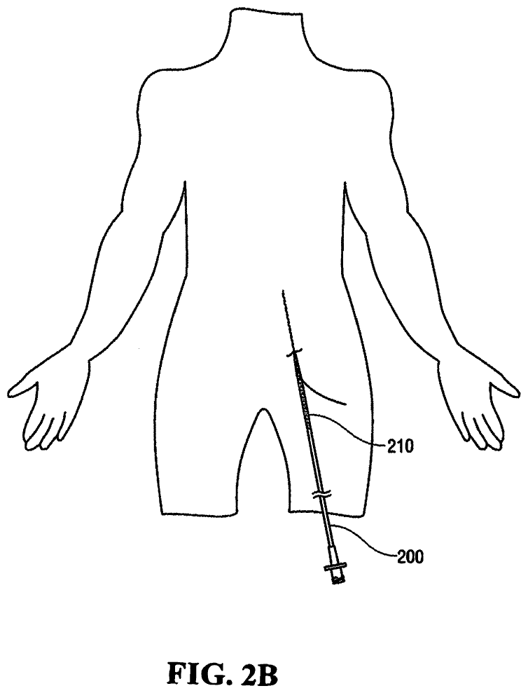

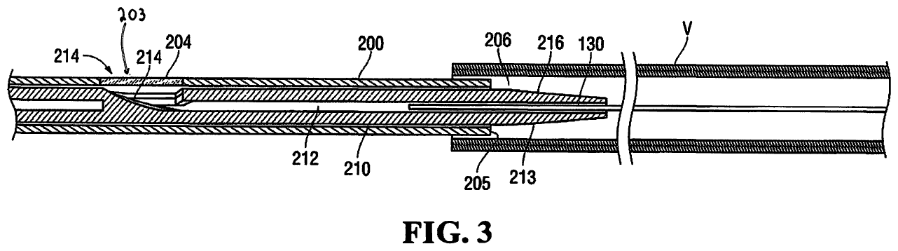

The catheter and dilator of the present invention can be inserted through various access ports in the body. FIG. 1A shows by way of example insertion of the short wire 130 through the radial artery for radial access and FIG. 2A shows insertion of the catheter and dilator over the short wire 130 of FIG. 1A; FIG. 1B shows by way of example an alternate insertion method through the femoral artery for femoral access and FIG. 2B shows insertion of the catheter and dilator over the short wire 130 of FIG. 1B. In either insertion method, or in insertion through other access ports (area) of the body, the catheter and dilator are used in the method depicted in FIGS. 3-10.

After the short wire 130 is inserted into the body, a proximal portion extends out from the skin S. Catheter 200, with the dilator 210 positioned concentrically therein (within a lumen of the catheter), is inserted over the exposed proximal end of the short wire 130 and advanced over the wire 130 into the vessel V as the wire 130 extends through the lumen 212 of the dilator 210 (see FIG. 3). Note the ramp 214 of the dilator 210 is aligned with the side port 202 of the catheter 200 (FIG. 4A). The dilator 210 extends past the distal edge 205 of the catheter 200 so at least the tip 213 is distal of the distal edge 205. The tip 213 is preferably conically dilating as illustrated, although other configurations are also contemplated.

As the dilator 210 and catheter 200 are advanced a certain distance, the proximal portion of the wire 130 will contact ramp 214 of the dilator 212. This ramp 214, as noted above, is aligned with the rapid exchange port (opening) 203 of catheter 200, which can be achieved by engagement or interlocking of the dilator 210 and catheter 200 in this aligned position or by an indicator or alignment markers. Port 203 has a seal 204 which can be in the form of any of the seals described herein-for illustrative purposes the seal is in the form of an elastic membrane overlying the port. As the dilator 210 and catheter 200 are further advanced over the short wire 130, the proximal end of the wire contacts ramp 214 which directs the wire 130 outwardly away from the longitudinal axis as shown in FIG. 4A. In preferred embodiments, the ramp remains outside the skin S so the wire 130 emerges outside the skin as shown. This ramp 214 forces the proximal end of the wire 130 to contact and move the seal 204 out of the way (open the seal) so the wire 130 moves past the seal 204 and out of the opening 203 in catheter 200 (see FIG. 4A). Note the wire 130 exits at a proximal end of the seal through gap 260 but alternatively could exit at a distal end of the seal or exit at other regions of the seal, e.g., side regions.

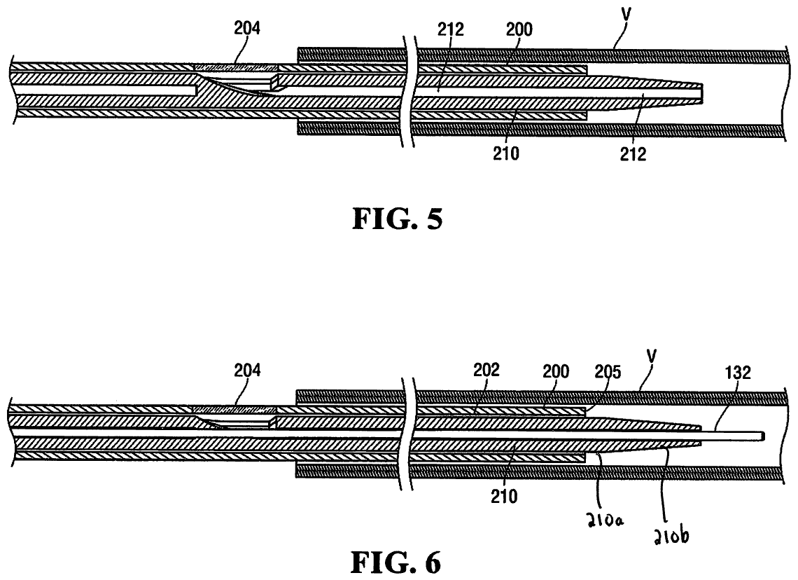

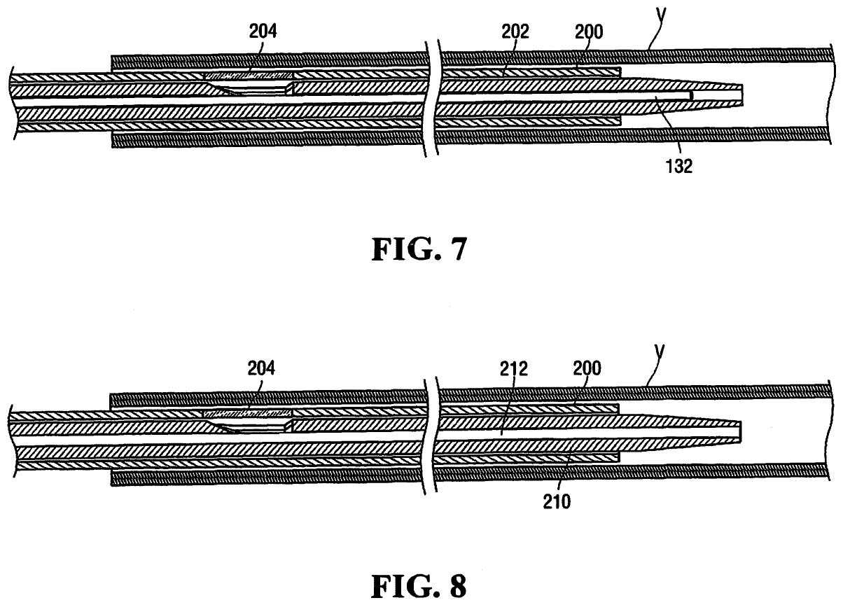

Next, the short wire 130 is removed (FIG. 4B) by withdrawing wire 130 proximally from lumen 212 leaving the catheter 200 and dilator 210 in place (FIG. 5). A longer guidewire 132 is then inserted through a proximal end of the catheter 200 and the dilator 210 (in embodiments where the dilator has a through lumen) and advanced distally within lumen 212 of the dilator 210 and preferably past the distal edge 205 of catheter 200. The catheter 200 and dilator 210 are then advanced distally over the long wire 132 (FIG. 6) to the target site. The guidewire 132 is then retracted (FIG. 7) and fully removed from the dilator 210 (FIG. 8). In an alternate embodiment, the dilator 210 is removed after the short wire 130 is removed and the long guidewire 132 is inserted through the lumen 206 of the catheter 200 instead of through the lumen 212 of the dilator 210. Note in some embodiments the dilator does not have an inner lumen proximal to the ramp such that the lumen only extends from the distal opening to the ramp portion. In such embodiments, the dilator is removed prior to insertion of the longer guidewire. Thus, FIGS. 4A, 4B and 6 show a version of the dilator with the lumen interrupted so the dilator does not have a through lumen. FIGS. 6-8 show the dilator with a through lumen. Except for passage of the long wire (after removal of the dilator for the dilator of FIG. 4A or optional removal of the dilator for FIG. 6), the methods of use are the same.

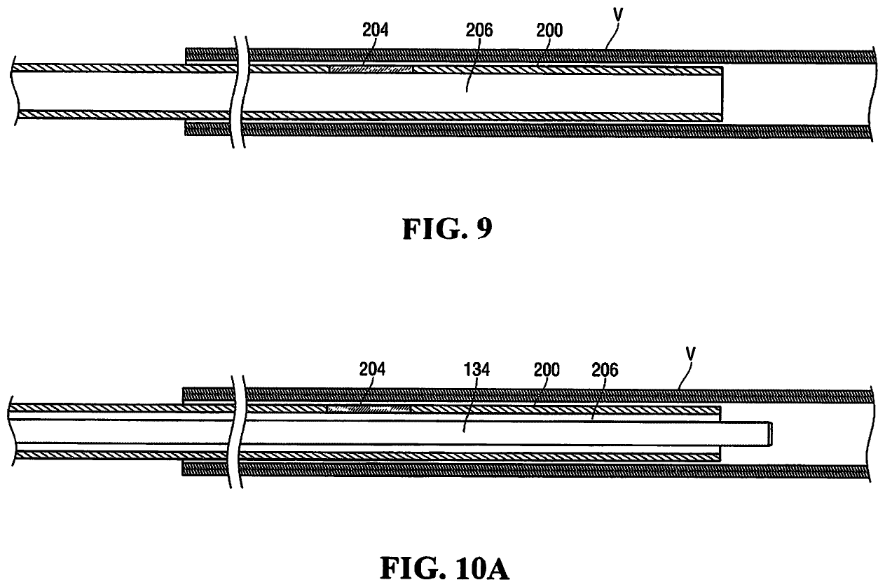

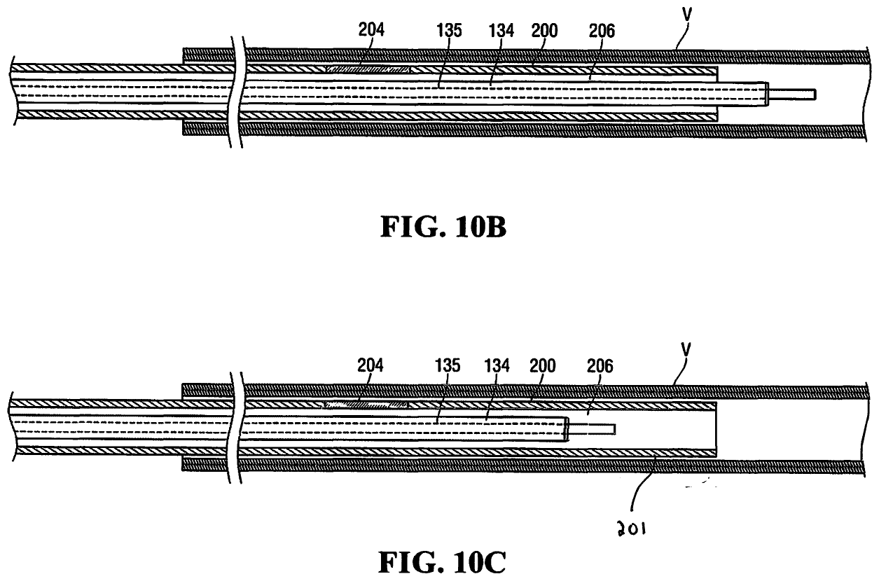

The dilator 210 is removed from lumen 206 of the catheter 200 as shown in FIG. 9 and an instrument 134 can be inserted through the lumen 206 of catheter 200 as shown in FIG. 10A. The instrument can include any endovascular device, for example a catheter, a catheter and a wire which the catheter can be further advanced over, a wire, a treatment device, a tissue removal device, an infusion device, a stent delivery device, a balloon catheter, etc. Multiple instruments can be exchanged and inserted through the catheter 200, coaxially and/or separately.

The short wire 130 can have a length ranging from about 10 cm to about 60 cm while the wire 132 can have a longer length ranging from about 70 cm to about 300 cm. Other lengths outside these ranges are contemplated for each wire as well.

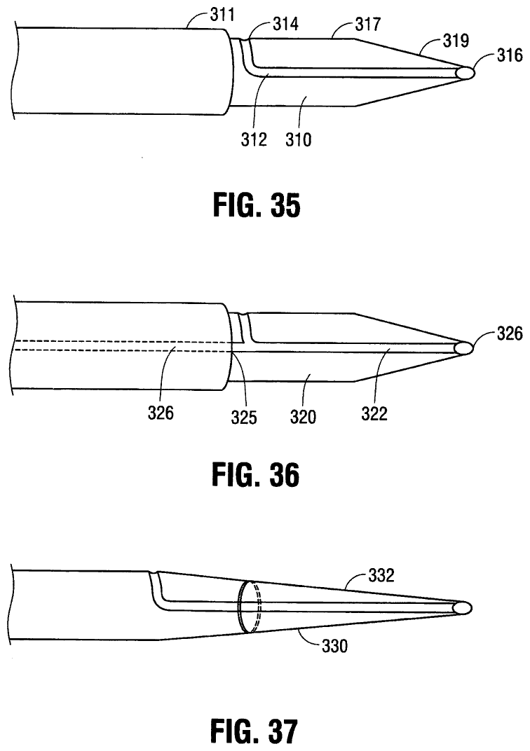

As noted above, in some embodiments there is no extension of the lumen of the dilator beyond the side hole, i.e., the lumen ends in the side hole, and a wire could not be passed from the end-hole. For example, dilator 310 of FIG. 35 has a lumen 312 which is primarily for the wire as it extends from the side hole 314 to the distal opening (end hole) 316. Side hole 314 can align with the rapid exchange port of the catheter and the hole 314 in some embodiments can be sealed or substantially self-sealing in the same manner as the seals described above for the catheter rapid exchange port as described above. The dilator has a non-tapered segment 317 that extends beyond the catheter 311 before a distal taper 319. In FIG. 36, the dilator 330 has a tapered section 332 extending beyond the catheter tip. The dilator extends from both ends of the catheter and there is a smooth transition between the dilator and the outside of the distal end of the catheter.

In the embodiment of FIG. 36, dilator 320 has a lumen 322 primarily for the wire extending from side hole 324 to distal opening 326. The lumen extends proximal to the ramp and side hole at region 325, but is dimensioned so the wire cannot pass into the lumen, e.g., has a smaller diameter at region 326, or has a partial blockage, to block wire passage while still in some embodiments enabling fluids to pass through. The lumen can alternatively be interrupted as shown in FIG. 5. With the smaller diameter lumen section, the wire which has a larger diameter cannot advance into the lumen so it will be forced from the distal dilator distal lumen out of the side hole.

If a proximal lumen is provided in the dilator (i.e., proximal to side hole and ramp), e.g., lumen 322, the catheter side hole can optionally be self-sealing or substantially sealing so that fluids/medication can be injected from a proximal end hole of the dilator, through the dilator and out its distal end hole, with minimal (if any) leakage through the side hole.

One of the goals of the dilator side hole is to be able to use a shorter wire to access the artery, e.g., radial artery. An access needle is advanced percutaneously into the vessel. A wire is advanced through the needle (optionally a small incision is made abutting the needle with a blade/knife). The needle is removed and the wire left in. The dilator (with catheter over the dilator) is advanced over the wire into the catheter. Then the wire is removed. This provides dilator access (optionally with catheter) into access artery such as the radial artery.

The side hole on the dilator can be between about 2 cm to about 30 cm from the distal end, although other distances are also contemplated. The dilator side hole is optionally “self sealing” as described above. The dilator side hole can be positioned distal of the catheter end hole upon initial insertion into the patient.

Most often the dilator is removed with the wire when the catheter distal end hole is still in the radial artery, then a radial cocktail can be infused. Subsequently, the additional length of the catheter/sheath e.g., a steerable catheter, can be advanced further, optionally over another inner catheter with an inner wire within the inner catheter (e.g., over catheter 134 and wire 135) until the outer catheter (e.g., catheter 200 of FIG. 10C) is in a place where the clinician wants to steer it. The inner catheter and wire are preferably either completely removed or at least withdrawn proximal to the steering zone(s) to allow the steering to function optimally. Such withdrawal to allow a distal section 201 to be steered is shown in FIG. 10C. The extent of withdrawal is preferably at least to a position proximal of the steering zone(s) so as not to interfere with deflection.

The catheters disclosed herein can be steerable and be provided with bending zones for bending at obtuse, acute, or right angles. The catheters or portions thereof can be made of shape-memory metals or polymers. Shape memory polymers can include for example, meth-acrylates, polyurethanes, blends of polystyrene and polyurethane, and PVC. Shape memory metals can include shape memory alloys (SMA) such as nickel-titanium (i.e., nitinol) by way of example.

In preferred transfemoral catheter embodiments, vascular fulcrums can optionally be used for support of the devices to reduce potential complications and risks.

The catheters and wires described herein can have tapered or non-tapered distal ends. They may have round or other shaped inner and/or outer circumferential configurations e.g., oval in cross-section.

The dilator in preferred embodiments has a non-tapered segment that extends beyond the catheter before the distal tapered segment. This is shown for example in FIG. 6 wherein dilator 210 has non-tapered segment 210a and tapered segment 210b.

The various approaches/accesses for the wires and catheters can be right femoral artery access and/or left femoral artery, radial, brachial, or axillary arterial access, veins, as well as other percutaneous ports of access. The devices can be used via non-percutaneous routes as well.

The catheters disclosed herein can be used in some embodiments by way of example to obviate the need for open surgical cutdowns of the common carotid artery (CCA) with a carotid stent, employing a percutaneous technique and carotid access devices which use anatomical fulcrums for added support.

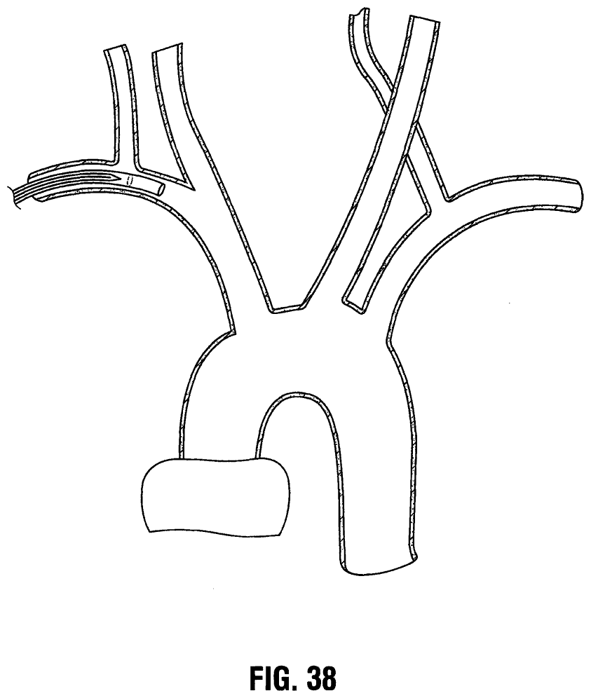

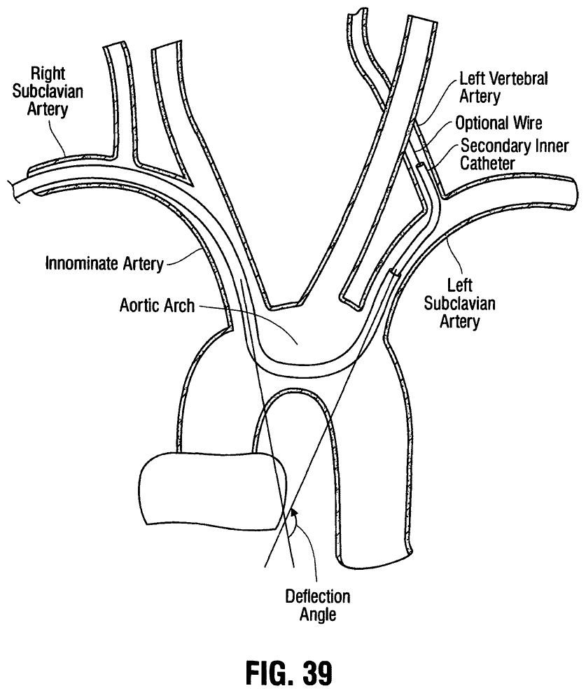

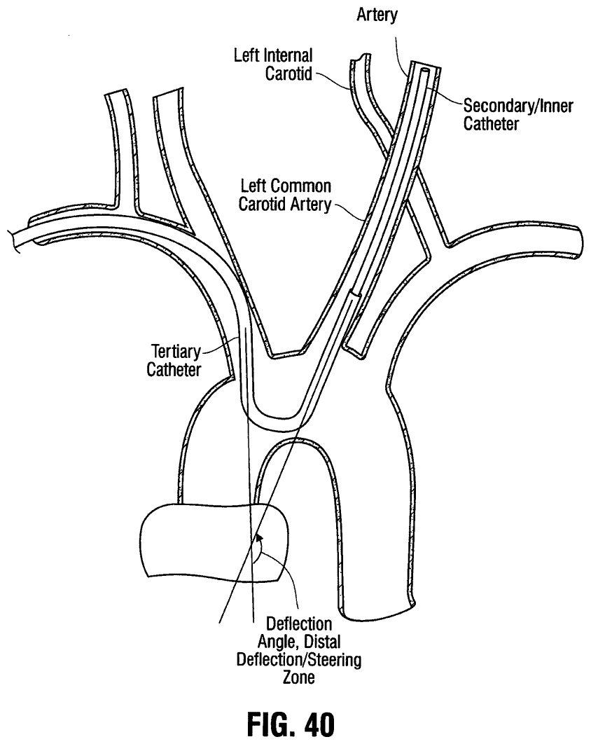

Various methods of use of the catheter and dilator of the present invention are described in the forgoing Summary Section of this application which lists method steps of various embodiments, and various alternatives and additions to the methods. For brevity, these are not repeated in this detailed description section. FIGS. 39 and 40 illustrate examples of methods wherein FIG. 38 shows insertion into the radial artery, FIG. 39 shows insertion into the left vertebral artery and FIG. 40 shows insertion into the left internal carotid artery. The deflection angle of the catheter is shown in the Figures.

Note the proximal and distal deflection zones are also referred to herein as proximal and distal steering zones.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, between the upper and lower limit of that range is encompassed within the invention.

Although the apparatus and methods of the subject invention have been described with respect to preferred embodiments, which constitute non-limiting examples, those skilled in the art will readily appreciate that changes and modifications may be made thereto without departing from the spirit and scope of the present invention as defined by the appended claims.

Additionally, persons skilled in the art will understand that the elements and features shown or described in connection with one embodiment may be combined with those of another embodiment without departing from the scope of the present invention.

Throughout the present disclosure, terms such as “approximately,” “about,” “generally,” “substantially,” and the like should be understood to allow for variations in any numerical range or concept with which they are associated. For example, it is intended that the use of terms such as “approximately,” “about” and “generally” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

In the claims:

1. A catheter comprising a proximal opening, a distal opening, a lumen extending through the catheter and a side opening forming a rapid exchange port dimensioned and configured for insertion of a guiding device through the side opening and a seal, the seal movable between a closed position to reduce leakage through the side opening and an open position, wherein the seal is movable to an open position by the guiding device.

2. The catheter of claim 1, wherein the guiding device is a guidewire.

3. The catheter of claim 1 in combination with a dilator, the dilator positioned within the lumen of the catheter and having a guiding surface adjacent the side opening, the guiding surface guiding the guiding device toward the side opening of the catheter.

4. The catheter of claim 3, wherein the guiding surface comprises a ramped surface angled away from the longitudinal axis toward the side opening.

5. The catheter of claim 1, wherein the seal comprises a tube having a side opening and connecting a first portion of the catheter with a second portion of the catheter, and an elastic material placed over the tube, wherein the guiding device presses against the elastic material to move the elastic material to provide a gap for exit of the guiding device through the side opening of the catheter.

6. The catheter of claim 1, wherein the catheter comprises a proximal body portion and a distal body portion, the proximal body portion is connected to the distal body portion by a connecting tube, wherein the seal is formed in the connecting tube.

7. The catheter of claim 6, further comprising an elastic material positioned over an opening in the connecting tube, a portion of the elastic material movable toward the side opening by the guiding device to create a gap for exit of the guiding device from the lumen.

8. The catheter of claim 1, wherein the seal is formed in a tube positioned in the lumen of the catheter, the tube having a coil embedded in the wall.

9. The catheter of claim 1, wherein the guiding device is configured to extend through a dilator positioned in the lumen of the catheter.

10. The catheter of claim 1, wherein the seal comprises a flap movably positioned over the opening and movable by the guiding device for exit of the guiding device through the side opening.

11. The catheter of claim 10, further comprising a band positioned on the catheter and an attachment element attaching the flap to the band, wherein the guiding device presses the flap outwardly away from the side opening to create a gap for exit from the lumen and the side opening and a tube having a band portion and a flap connected to the band portion, the tube positioned in the side opening.

12. The catheter of claim 1, wherein the catheter further comprises an inner polymeric liner, the liner movable by the guiding device to create a gap for exit of the guiding device through the side opening.

13. The catheter of claim 1, wherein the catheter has a distal deflection zone deflectable by axial movement of a first wire.

14. The catheter of claim 13, wherein the catheter has a proximal deflection zone positioned proximal of the distal zone and deflectable by a second wire.

15. The catheter of claim 13, wherein the first wire is offset from a longitudinal axis of the catheter and is attached to a pull ring, and pulling of the first wire deflects the distal deflection zone initially in the direction of the first wire, wherein the pull ring and first wire are substantially within a wall of the catheter.

16. The catheter of claim 13, wherein the first wire is a push wire offset from a longitudinal axis of the catheter and pushing of the first wire deflects the distal deflection zone initially opposite the first wire.

17. The catheter of claim 14, further comprising a distal pull ring and a proximal pull ring, and the first wire attached to the distal pull ring and a second wire is attached to the proximal pull ring, the proximal and distal deflection zones being independently deflectable.

18. (canceled)

19. A system comprising:
a) a catheter having proximal opening, a distal opening, a lumen extending through the catheter and a side opening forming a rapid exchange port dimensioned and configured for insertion of a guiding device through the side opening; and
b) a dilator, the dilator positioned within the lumen of the catheter and having a guiding surface adjacent the side opening, the guiding surface guiding the guiding device toward the side opening of the catheter.

20. The system of claim 19, wherein the guiding surface comprises a ramped surface angled away from the longitudinal axis toward the side opening.

21. (canceled)

22. The system of claim 19, wherein the catheter has a first and second independently deflectable detachment zones, wherein the first and second detachment zones each have a pull ring and a pull wire connected to the pull ring.

23. (canceled)

24-64. (canceled)
